(19) United States
(12) Patent Application Publication
(10) Pub. No.: US 2023/0227813 A1
(43) Pub. Date: Jul. 20, 2023

(54) PARALLEL ANALYSIS OF INDIVIDUAL CELLS FOR RNA EXPRESSION AND DNA FROM TARGETED TAGMENTATION BY SEQUENCING

(71) Applicant: Ludwig Institute for Cancer Research Ltd, Zurich (CH)

(72) Inventors: Bing Ren, La Jolla, CA (US); Chenxu Zhu, La Jolla, CA (US)

(73) Assignee: Ludwig Institute for Cancer Research Ltd, Zurich (CH)

(21) Appl. No.: 18/001,898

(22) PCT Filed: Jun. 22, 2021
(86) PCT No.: PCT/US21/38409

§ 371 Date: Dec. 15, 2022

Related U.S. Application Data
(60) Provisional application No. 63/042,761, filed on Jun. 23, 2020.

(51) Int. Cl. C12N 15/10

(52) U.S. Cl. CPC C12N 15/1072

(57) ABSTRACT

The present invention relates to methods for the joint analysis of regulation of gene expression and gene expression in single cells. Provided are methods for obtaining gene expression information for a single nucleus, the methods comprising deriving a DNA library from the genomic DNA in one or more nuclei and deriving an RNA library from the RNA in one or more nuclei, sequencing the molecules in the RNA library and the DNA library, and correlating the RNA library and the DNA library for each of the one or more nuclei.

### STATEMENT REGARDING FEDERALLY SPONSORED RESEARCH

This invention was made with government support under 1U19 MH114831-02 (awarded by the National Institute of Mental Health (NIMH)), under U01MH121282 (awarded by the NIMH), and RO1AG066018 (awarded by the National Institute of Aging). The government has certain rights in the invention.

### FIELD OF THE INVENTION

The present invention relates to methods for the joint analysis of regulation of gene expression and gene expression in single cells.

### BACKGROUND

In a multi-cellular organism, virtually every cell type contains an identical copy of the same genetic material. However, the epigenome, including the state of DNA methylation and histone modifications, differs substantially between cell types. The epigenome plays a critical role in gene regulation in a number of ways—by organizing the nuclear architecture of the chromosomes, restricting or facilitating transcription factor access to DNA, preserving a memory of past transcriptional activities, and fine-tuning the abundance of protein-coding mRNA sequences in the cell. A comprehensive view of the epigenome in each cell type is crucial for delineating the gene regulatory programs in different cell lineages during development and in pathological conditions. However, different histone modifications can vary greatly in their cellular specificity and relationships to cell-type-specific gene expression, leading to varying degrees of success in resolving cellular heterogeneity from complex tissues. This makes it very challenging or nearly impossible to integrate datasets of different histone marks from different experiments. Moreover, to better understand the gene regulatory mechanisms, it is necessary to assess the transcriptional profiles along with chromatin states from the same cells. Thus, a single-cell approach that can jointly assay both chromatin state and gene expression would be highly desired.

### SUMMARY OF THE INVENTION

In one aspect, provided is a method for obtaining gene expression information for a single nucleus, the method comprising:
a. permeabilizing one or more nuclei;
b. contacting the one or more nuclei with (i) an antibody that binds to a chromatin-associated protein or chromatin modification and (ii) a first transposase; wherein the first transposase is loaded with a nucleic acid comprising a first tag, wherein the first tag comprises a first restriction site and a barcode selected from a first set of barcodes;
c. initiating a tagmentation reaction, resulting in the generation of genomic DNA fragments comprising the first tag;
d. reverse transcribing the RNA in the one or more nuclei using primers comprising a second tag, wherein the second tag comprising a second restriction site and the barcode of the first tag, resulting in the generation of cDNA comprising the second tag;
e. contacting the one or more nuclei with a ligase and a third tag comprising a second barcode selected from a second set of barcodes, resulting in the generation of genomic DNA fragments comprising a first tag and a third tag and cDNA comprising a second tag and a third tag;
f. lysing the one or more nuclei;
g. fusing a polynucleotide tail to the DNA and cDNA, generating polynucleotide tailed DNA and cDNA;
h. amplifying the polynucleotide tailed DNA and cDNA, wherein one of the primers used for the amplification of the DNA comprises a third restriction site and wherein the third restriction site is recognized by an endonuclease;
i. dividing the amplified polynucleotide tailed DNA and cDNA into a DNA library and an RNA library;
j. for the DNA library:
   i. cleaving the amplified polynucleotide tailed DNA with a restriction an endonuclease recognizing the third restriction site;
   ii. contacting the DNA end with a sequencing adaptor and a ligase, resulting in the generation of amplified polynucleotide tailed DNA comprising the sequencing adaptor;
   iii. cleaving the amplified polynucleotide tailed cDNA with an enzyme recognizing the second restriction site;
k. for the RNA library:
   i. cleaving the amplified polynucleotide tailed DNA with a restriction enzyme recognizing the first restriction site; ii. contacting the amplified polynucleotide tailed cDNA with a second transposase loaded with a nucleic acid comprising a sequencing adaptor and initiating a tagmentation reaction, resulting in the generation of amplified polynucleotide tailed cDNA comprising the sequencing adaptor; 1. sequencing the molecules in the RNA library and the DNA library; m. correlating the RNA library and the DNA library for each of the one or more nuclei.

In one aspect, provided is a method for obtaining gene expression information for a single nucleus, the method comprising:
a. permeabilizing one or more nuclei;
b. contacting the one or more nuclei with (i) an antibody that binds to a chromatin-associated protein or chromatin modification and (ii) a first transposase; wherein the first transposase is loaded with a nucleic acid comprising a first tag, wherein the first tag comprises a first restriction site and a barcode selected from a first set of barcodes;
c. initiating a tagmentation reaction, resulting in the generation of genomic DNA fragments comprising the first tag;
d. reverse transcribing the RNA in the one or more nuclei using primers comprising a second tag, wherein the second tag comprising a second restriction site and the barcode of the first tag, resulting in the generation of cDNA comprising the second tag;
e. contacting the one or more nuclei with a ligase and a third tag comprising a second barcode selected from a second set of barcodes, resulting in the generation of genomic DNA fragments comprising a first tag and a third tag and cDNA comprising a second tag and a third tag;
f. lysing the one or more nuclei;
g. fusing a polynucleotide tail to the DNA and cDNA, generating polynucleotide tailed DNA and cDNA;
h. amplifying the polynucleotide tailed DNA and cDNA, wherein one of the primers used for the amplification of the cDNA comprises a third restriction site and wherein the third restriction site is recognized by an endonuclease;
i. dividing the amplified polynucleotide tailed DNA and cDNA into a DNA library and an RNA library;
j. for the RNA library:
   i. cleaving the amplified polynucleotide tailed cDNA with a restriction an endonuclease recognizing the third restriction site;
   ii. contacting the cDNA end with a sequencing adaptor and a ligase, resulting in the generation of amplified polynucleotide tailed cDNA comprising the sequencing adaptor;
   iii. cleaving the amplified polynucleotide tailed DNA with an enzyme recognizing the first restriction site;
k. for the DNA library:
   i. cleaving the amplified polynucleotide tailed cDNA with a restriction enzyme recognizing the second restriction site;
   ii. contacting the amplified polynucleotide tailed DNA with a second transposase loaded with a nucleic acid comprising a sequencing adaptor and initiating a tagmentation reaction, resulting in the generation of amplified polynucleotide tailed DNA comprising the sequencing adaptor;
l. sequencing the molecules in the RNA library and the DNA library; m. correlating the RNA library and the DNA library for each of the one or more nuclei.

In one aspect, provided is a method for obtaining gene expression information for a single nucleus, the method comprising:
a. permeabilizing one or more nuclei;
b. contacting the one or more nuclei with (ii) an antibody that binds to a chromatin-associated protein or chromatin modification and (ii) a first transposase; wherein the first transposase is loaded with a nucleic acid comprising a first tag,
   wherein the first tag comprises a first barcode selected from a first set of barcodes;
c. initiating a tagmentation reaction, resulting in the generation of genomic DNA fragments comprising the first tag;
d. reverse transcribing the RNA in the one or more nuclei using primers comprising a second tag, wherein the second tag comprises the barcode of the first tag, resulting in the generation of cDNA comprising the second tag;
   wherein the first tag further comprises (i) a first reactive group suitable to perform click chemistry or (ii) a first affinity tag and/or wherein the second tag further comprises (i) a second reactive group suitable to perform click chemistry or (ii) a second affinity tag;
e. contacting the one or more nuclei with a ligase and a third tag comprising a second barcode selected from a second set of barcodes, resulting in the generation of genomic DNA fragments comprising a first tag and a third tag and cDNA comprising a second tag and a third tag;
f. lysing the one or more nuclei;
g. (I) contacting the genomic DNA fragments with an immobilized agent that
   (i) reacts with the first reactive group; or
   (ii) binds to the first affinity tag; and performing a pull-down of the genomic DNA to separate the genomic DNA from the cDNA; and/or
(II) contacting the cDNA with an immobilized agent that
   (i) reacts with the second reactive group; or
   (ii) binds to the second affinity tag; and
   performing a pull-down of the cDNA to separate the genomic cDNA from the DNA;
h. for the DNA library:
   1. contacting the genomic DNA with random primers comprising a sequencing adaptor, generating polynucleotide tailed DNA; and
   2. amplifying the polynucleotide tailed DNA;
i. for the RNA library:
   1. contacting the cDNA with random primers comprising a sequencing adaptor, generating polynucleotide tailed cDNA; and
   2. amplifying the polynucleotide tailed cDNA;
j. sequencing the molecules in the RNA library and the DNA library;
k. correlating the RNA library and the DNA library for each of the one or more nuclei.

In one embodiment, for the step of contacting the one or more nuclei with (i) an antibody that binds to a chromatin-associated protein or chromatin modification and (ii) a first transposase: (i) the one or more nuclei are first contacted with the antibody and then contacted the first transposase, wherein the first transposase is linked to a binding moiety that binds to the antibody; (ii) the antibody is first incubated with the first transposase linked to a binding moiety that binds to the antibody; and the one or more nuclei are contacted with the antibody bound to the transposase; or (iii) the one or more nuclei are contacted with an antibody that is covalently linked to the first transposase.

In one embodiment, after the step of contacting the one or more nuclei with a ligase and a third tag comprising a second barcode selected from a second set of barcodes, the method further comprises a step of contacting the one or more nuclei with a ligase and a fourth tag comprising a third barcode selected from a third set of barcodes, resulting in the generation of genomic DNA fragments comprising a first, a third, and a fourth tag and in the generation of cDNA comprising a second, a third tag, and a fourth tag.

In some embodiments, the step of contacting the one or more nuclei with a ligase and a tag comprising an additional barcode is repeated one or more times. In some embodiments, the step of contacting the one or more nuclei with a ligase and a tag comprising an additional barcode is repeated 2, 3, 4, 5, 6, 7, 8, 9, or 10 times.

In one embodiment, the polynucleotide tail is fused to the DNA and cDNA by contacting the DNA and cDNA with a terminal deoxynucleotidyltransferase (TdT). In one embodiment, the polynucleotide tail is fused to the DNA and cDNA by contacting the DNA and cDNA with a DNA ligase and DNA or RNA oligonucleotide. In some embodiments, the DNA ligase is a T3, T4 or T7 DNA ligase. In one embodiment, the polynucleotide tail is fused to the DNA and cDNA by contacting the DNA and cDNA with a DNA polymerase and a random primer. In one embodiment, the polynucleotide tail is fused to the DNA and cDNA by contacting the DNA and cDNA with a DNA or RNA oligonucleotide with reactive chemical group that attaches to the 3′-end of the DNA and cDNA. In some embodiments, the reactive chemical group is an azide group or an alkyne group.

In one aspect, provided is a method for obtaining gene expression information for a single nucleus, the method comprising:
a. providing a sample comprising nuclei;
b. dividing the sample into a first set of sub-samples comprising two or more sub-samples;
c. permeabilizing the nuclei in the two or more sub-samples in the first set of sub-samples;
d. contacting the nuclei in the two or more sub-samples in the first set of sub-samples with (i) an antibody that binds to a chromatin-associated protein or chromatin modification and (ii) a first transposase;
   wherein the first transposase is loaded with a nucleic acid comprising a first tag comprising a barcode selected from a first set of barcodes;
e. initiating a tagmentation reaction, resulting in the generation of genomic DNA fragments comprising the first tag;
f. reverse transcribing the RNA in the one or more nuclei in the two or more sub-samples in the first set of sub-samples using primers comprising a second tag, wherein the second tag comprising a second restriction site and the barcode of the first tag, resulting in the generated of cDNA comprising the second tag;
g. pooling the first set of sub-samples to generate a first sub-sample pool;
h. dividing the first sub-sample pool into two or more sub-samples to generate a second set of sub-samples;
i. contacting each of the two or more sub-samples in the second set of sub-samples with a ligase and a third tag comprising a barcode selected from a second set of barcodes, wherein the third tag is ligated to the genomic DNA and the cDNA;
j. pooling the second set of sub-samples to generate a second sub-sample pool;
k. dividing the second sub-sample pool into two or more sub-samples to generate a third set of sub-samples;
l. contacting each of the two or more sub-samples in the third set of sub-samples with a ligase and a fourth tag comprising a barcode selected from a third set of barcodes, wherein the fourth tag is ligated to the genomic DNA and the cDNA;
m. pooling the two or more sub-samples in the third set of sub-samples;
n. lysing the nuclei;
o. fusing a polynucleotide tail to the DNA and cDNA, generating polynucleotide tailed DNA and cDNA;
p. amplifying the polynucleotide tailed DNA and cDNA, wherein one of the primers used for the amplification of the DNA comprises a third restriction site;
q. dividing the amplified polynucleotide tailed DNA and cDNA into a DNA library and a RNA library;
r. for the DNA library:
   1. cleaving the amplified polynucleotide tailed DNA with a restriction an endonuclease recognizing the third restriction site;
   2. contacting the DNA end with a sequencing adaptor and a ligase, resulting in the generation of amplified polynucleotide tailed DNA comprising the sequencing adaptor;
   3. cleaving the amplified polynucleotide tailed cDNA with an enzyme recognizing the second restriction site;
s. for the RNA library:
   1. cleaving the amplified polynucleotide tailed DNA with a restriction enzyme recognizing the first restriction site;
   2. contacting the amplified polynucleotide tailed cDNA with a second transposase loaded with a nucleic acid comprising a sequencing adaptor and initiating a tagmentation reaction, resulting in the generation of amplified polynucleotide tailed cDNA comprising the sequencing adaptor;
t. sequencing the RNA library and the DNA library;
u. correlating the RNA library and the DNA library for each of the one or more nuclei.

In one aspect, provided is a method for obtaining gene expression information for a single nucleus, the method comprising:
a. providing a sample comprising nuclei;
b. dividing the sample into a first set of sub-samples comprising two or more sub-samples;
c. permeabilizing the nuclei in the two or more sub-samples in the first set of sub-samples;
d. contacting the nuclei in the two or more sub-samples in the first set of sub-samples with (i) an antibody that binds to a chromatin-associated protein or chromatin modification and (ii) a first transposase;
   wherein the first transposase is loaded with a nucleic acid comprising a first tag comprising a barcode selected from a first set of barcodes;
e. initiating a tagmentation reaction, resulting in the generation of genomic DNA fragments comprising the first tag;
f. reverse transcribing the RNA in the one or more nuclei in the two or more sub-samples in the first set of sub-samples using primers comprising a second tag, wherein the second tag comprising a second restriction site and the barcode of the first tag, resulting in the generated of cDNA comprising the second tag;
g. pooling the first set of sub-samples to generate a first sub-sample pool;
h. dividing the first sub-sample pool into two or more sub-samples to generate a second set of sub-samples;
i. contacting each of the two or more sub-samples in the second set of sub-samples with a ligase and a third tag comprising a barcode selected from a second set of barcodes, wherein the third tag is ligated to the genomic DNA and the cDNA;
j. pooling the second set of sub-samples to generate a second sub-sample pool;
k. dividing the second sub-sample pool into two or more sub-samples to generate a third set of sub-samples;
l. contacting each of the two or more sub-samples in the third set of sub-samples with a ligase and a fourth tag comprising a barcode selected from a third set of barcodes, wherein the fourth tag is ligated to the genomic DNA and the cDNA;
m. pooling the two or more sub-samples in the third set of sub-samples;
n. lysing the nuclei;
o. fusing a polynucleotide tail to the DNA and cDNA, generating polynucleotide tailed DNA and cDNA;
p. amplifying the polynucleotide tailed DNA and cDNA, wherein one of the primers used for the amplification of the cDNA comprises a third restriction site;
q. dividing the amplified polynucleotide tailed DNA and cDNA into a DNA library and an RNA library;
r. for the RNA library:

1. cleaving the amplified polynucleotide tailed cDNA with a restriction an endonuclease recognizing the third restriction site; 2. contacting the cDNA end with a sequencing adaptor and a ligase, resulting in the generation of amplified polynucleotide tailed cDNA comprising the sequencing adaptor; 3. cleaving the amplified polynucleotide tailed DNA with an enzyme recognizing the first restriction site;
s. for the DNA library:
   1. cleaving the amplified polynucleotide tailed cDNA with a restriction enzyme recognizing the second restriction site;
   2. contacting the amplified polynucleotide tailed DNA with a second transposase loaded with a nucleic acid comprising a sequencing adaptor and initiating a tagmentation reaction, resulting in the generation of amplified polynucleotide tailed DNA comprising the sequencing adaptor;
t. sequencing the RNA library and the DNA library;
u. correlating the RNA library and the DNA library for each of the one or more nuclei.

In some embodiments, for the step of contacting the nuclei in the two or more sub-samples in the first set of sub-samples with (i) an antibody that binds to a chromatin-associated protein or chromatin modification and (ii) a first transposase: (i) the one or more nuclei in the two or more sub-samples are first contacted with the antibody and then contacted the first transposase, wherein the first transposase is linked to a binding moiety that binds to the antibody; (ii) the antibody is first incubated with the first transposase linked to a binding moiety that binds to the antibody; and the one or more nuclei in the two or more sub-samples are contacted with the antibody bound to the transposase; (iii) the one or more nuclei in the two or more sub-samples are contacted with an antibody that is covalently linked to the first transposase.

In some embodiments, after the step of pooling the two or more sub-samples in the third set of sub-samples, the method further comprises repeating the steps of pooling; dividing; and contacting the sub-samples with a ligase and a tag comprising an additional barcode one or more times. In some embodiments, after the step of pooling the two or more sub-samples in the third set of sub-samples, the method further comprises repeating the steps of pooling; dividing; and contacting the sub-samples with a ligase and a tag comprising an additional barcode 2, 3, 4, 5, 6, 7, 8, 9, or 10 times.

In some embodiments, the third restriction site is recognized by a type IIS endonuclease. In some embodiments, the IIS endonuclease is selected from the group consisting of FokI, AcuI, AsuHPI, BbvI, BpmI, BpuEI, BseMII, BseRI, BseXI, BsgI, BslFI, BsmFI, BsPCNI, BstV1I, BtgZI, EciI, Eco57I, FaqI, GsuI, HphI, MmeI, NmeAIII, SchI, TaqII, TspDTI, TspGWI. On one embodiment, the type IIS endonuclease is FokI.

In one embodiment, the polynucleotide tail is fused to the DNA and cDNA by contacting the DNA and cDNA with a terminal deoxynucleotidyltransferase (TdT). In one embodiment, the polynucleotide tail is fused to the DNA and cDNA by contacting the DNA and cDNA with a DNA ligase and DNA or RNA oligonucleotide. In some embodiments, the DNA ligase is a T3, T4 or T7 DNA ligase. In one embodiment, the polynucleotide tail is fused to the DNA and cDNA by contacting the DNA and cDNA with a DNA polymerase and a random primer. In one embodiment, the polynucleotide tail is fused to the DNA and cDNA by contacting the DNA and cDNA with a DNA or RNA oligonucleotide with reactive chemical group that attaches to the 3′-end of the DNA and cDNA. In some embodiments, the reactive chemical group is an azide group or an alkyne group. In some embodiments, the reactive chemical group is reactive group suitable to perform click chemistry.

In one embodiment, the binding moiety linked to the first transposase is protein A.

In some embodiments, the chromatin-associated protein is a histone protein, transcription factor, chromatin remodeling complex, RNA polymerase, DNA polymerase, or accessory proteins.

In some embodiments, the chromatin modification is a histone modification, DNA modification, RNA modifications, histone variants, or DNA structure that can be recognized by an antibody such as R-loop.

In one embodiment, the nuclei are obtained from a mammal.

### BRIEF DESCRIPTION OF THE DRAWINGS

FIG. 1 illustrates the Paired-Tag workflow. Nuclei were first stained with antibodies targeting different histone marks; targeted tagmentation and reverse transcription were then performed. Two rounds of ligation-based combinatorial barcoding enable the labelling of hundreds of thousands single nuclei. The resulting DNA is then PCR amplified and separated for the detection of histone modifications and gene expression.

FIG. 2 illustrates the second adaptor tagging of DNA and RNA libraries. For DNA libraries, amplified products were digested with a type IIS restriction enzyme-FokI, and the cohesive end was then used to ligate the P5 adaptor. For RNA libraries, N5 adaptor was added by tagmentation.

FIGS. 3A, 3B, 3C, and 3D illustrate a sequential incubation protocol. FIG. 3A. Schematics for two strategies. Sequential incubation: nuclei were first extracted and stained with antibodies overnight; in Day2, nuclei were first washed three times and incubated with pA-Tn5 for 1 hr, followed by a second washing for three times and tagmentation reactions were then initiated. Pre-incubation: during the preparation of nuclei, pA-Tn5 and antibodies were first pre-incubated for lhr and the antibody-pA-Tn5 complexes were then incubated with nuclei overnight; in Day2, nuclei were washed for three times and tagmentation reactions were then initiated. FIG. 3B. Scatter plot showing the number of raw sequenced reads per nuclei and the corresponding number of unique loci per nuclei for single cells. Cells from sequential incubation and pre-incubation experiments are shown. FIG. 3C. Violin plots showing fraction of reads inside peaks for single cells from sequential incubation and pre-incubation experiments. FIG. 3D. Genome browser view showing the aggregated H3K27me3 signals for representative regions from sequential incubation and pre-incubation experiments. ENCODE H3K27me3 ChIP-seq data are also shown for reference.

FIG. 4 illustrates one way of separating DNA and RNA libraries.

### DETAILED DESCRIPTION OF THE INVENTION

The disclosure provides methods for the joint analysis of regulation of gene expression and gene expression in single cells. The analysis of gene expression regulation may include the analysis of the interaction patterns of a protein involved in the regulation of gene expression, such as the binding of a chromatin-associated protein to a sequence of DNA and/or may include an analysis of the pattern of an epigenetic chromatin modification of interest (including histone or DNA modifications).

In one embodiment, provided is a high-throughput method comprising: (1) targeted tagmentation of specific chromatin regions with one or more protein A-fused transposases guided by antibodies that specifically bind to chromatin-associated protein or epigenetic chromatin modification of interest, (2) simultaneously labeling both cDNA from reverse transcription (RT) and chromatin DNA from targeted tagmentation with a ligation-based combinatorial barcoding strategy, and (3) generation of separate sequencing libraries to profile each molecular modality.

Transposase-Mediated Tagmentation

Provided herein are methods for the joint analysis of regulation of gene expression and gene expression in a single cell or populations of cells. The analysis of gene expression regulation may include the analysis of the interaction patterns of a protein involved in the regulation of gene expression, such as the binding of a chromatin-associated protein to a sequence of DNA, and/or may include an analysis of the pattern of an epigenetic chromatin modification of interest.

As used herein, chromatin-associated proteins are proteins that can be found at one or more sites on the chromatin and/or that may associate with chromatin in a transient manner. Examples of chromatin-associated factors include, but are not limited to, transcription factors (e.g., tumor suppressors, oncogenes, cell cycle regulators, development and/or differentiation factors, general transcription factors (TFs)), DNA and RNA polymerases, components of the transcriptional machinery, ATP-dependent chromatin remodelers (e.g., (P)BAF, MOT1, ISWI, IN080, CHD1), chromatin remodeling proteins (e.g., histone acetyl transferase (HAT)) complexes, histone deacetylase (HDAC)) histone methylases/demethylases, SWI/SNF complexes, NURD), DNA methyltransferases (DNMT1, DNMT3A/B), replication factors and the like. Such proteins may interact with the chromatin (DNA, histones) at particular phases of the cell cycle (e.g., G1, S, G2, M-phase), upon certain environmental cues (e.g., growth and other stimulating signals, DNA damage signals, cell death signals), upon transfection and transient or stable expression (e.g., recombinant factors) or upon infection (e.g., viral factors). Chromatin-associated proteins also include histones and their variants. Histones may be modified at histone tails through posttranslational modifications which alter their interaction with DNA and nuclear proteins and influence for example gene regulation, DNA repair and chromosome condensation. The H3 and H4 histones have long tails protruding from the nucleosome which can be covalently modified, for example by methylation, acetylation, phosphorylation, ubiquitination, sumoylation, citrullination and ADP-ribosylation. The core of the histones H2A and H2B can also be modified.

In some embodiments, the binding of the chromatin-associated factor to the sequence of chromatin DNA is direct. In other words, the chromatin-associated factor makes direct contacts with the chromatin DNA and is in direct physical contact with the chromatin DNA, as it would be the case with DNA binding transcription factors. In other embodiments, the binding of the chromatin-associated factor of interest to the sequence of chromatin DNA is indirect. In other words, the contact may be indirect, such as through the members of a complex.

In some embodiments, the disclosed methods are used for analyzing the binding of transcription factors to a sequence of DNA in a single cell (or a population of cells). As used herein, a transcription factor is a protein that affects regulation of gene expression. In particular, transcription factors regulate the binding of RNA polymerase and the initiation of transcription. A transcription factor binds upstream or downstream to either enhance or repress transcription of a gene by assisting or blocking RNA polymerase binding. The term transcription factor includes both inactive and activated transcription factors. Exemplary transcription factors include but are not limited to AAF, abb1, ADA2, ADA-NF1, AF-1, AFP1, AhR, AIIN3, ALL-1, alpha-CBF, alpha-CP 1, alpha-CP2a, alpha-CP2b, alphaHo, alphaH2-alphaH3, Alx-4, aMEF-2, AML1, AML1a, AML1b, AML1c, AML1DeltaN, AML2, AML3, AML3a, AML3b, AMY-1L, A-Myb, ANF, AP-1, AP-2alphaA, AP-2alphaB, AP-2beta, AP-2gamma, AP-3 (1), AP-3 (2), AP-4, AP-5, APC, AR, AREB6, Arnt, Amt (774 M form), ARP-1, ATBF1-A, ATBF1-B, ATF, ATF-1, ATF-2, ATF-3, ATF-3deltaZIP, ATF-a, ATF-adelta, ATPF1, Bar1111, Barh12, Barxl, Barx2, Bc1-3, BCL-6, BD73, beta-catenin, Binl, B- Myb, BP1, BP2, brahma, BRCA1, Brn-3a, Brn-3b, Brn-4, BTEB, BTEB2, B-TFIID, C/EBPalpha, C/EBPbeta, C/EBPdelta, CACCbinding factor, Cart-1, CBF (4), CBF (5), CBP, CCAAT-binding factor, CCMT-binding factor, CCF, CCG1, CCK-la, CCK-lb, CD28RC, cdk2, cdk9, Cdx-1, CDX2, Cdx-4, CFF, Chx10, CLIMI, CLIM2, CNBP, CoS, COUP, CPI, CPIA, CPIC, CP2, CPBP, CPE binding protein, CREB, CREB-2, CRE-BPI, CRE-BPa, CREMalpha, CRF, Crx, CSBP-1, CTCF, CTF, CTF-1, CTF-2, CTF-3, CTF-5, CTF-7, CUP, CUTL1, Cx, cyclin A, cyclin Tl, cyclin T2, cyclin T2a, cyclin T2b, DAP, DAXL DB1, DBF4, DBP, DbpA, DbpAv, DbpB, DDB, DDB-1, DDB-2, DEF, deltaCREB, deltaMax, DF-1, DF-2, DF-3, Dlx-1, Dlx-2, Dlx-3, DIx4 (long isoform), Dlx-4 (short isoform, Dlx-5, Dlx-6, DP-1, DP-2, DSIF, DSIF-p14, DSIF-p160, DTF, DUX1, DUX2, DUX3, DUX4, E, El 2, E2F, E2F+E4, E2F+p107, E2F-1, E2F-2, E2F-3, E2F-4, E2F-5, E2F-6, E47, E4BP4, E4F, E4F1, E4TF2, EAR2, EBP-80, EC2, EF1, EF-C, EGR1, EGR2, EGR3, EIIaE-A, EIIaE-B, EIIaE-Calpha, EIIaE-Cbeta, EivF, EIf-1, EIk-1, Emx-1, Emx-2, Emx-2, En-1, En-2, ENH-bind. prot, ENKTF-1, EPAS1, epsilonFl, ER, Erg-1, Erg-2, ERR1, ERR2, ETF, Ets-1, Ets-1 deltaVil, Ets-2, Evx-1, F2F, factor 2, Factor name, FBP, f-EBP, FKBP59, FKHL18, FKHRL1P2, Fli-1, Fos, FOXB1, FOXCl, FOXC2, FOXD1, FOXD2, FOXD3, FOXD4, FOXEL FOXE3, FOXF1, FOXF2, FOXG1a, FOXG1b, FOXG1c, FOXH1, FOXI1, FOXJ1a, FOXJ1b, FOXJ2 (long isoform), FOXJ2 (short isoform), FOXJ3, FOXKIa, FOXKIb, FOXKlc, FOXL1, FOXMla, FOXMlb, FOXM1c, FOXN1, FOXN2, FOXN3, FOX01a, FOX01b, FOX02, FOX03a, FOX03b, FOX04, FOXP1, FOXP3, Fra-1, Fra-2, FTF, FTS, G factor, G6 factor, GABP, GABP-alpha, GABP-betal, GABP-beta2, GADD 153, GAF, gammaCMT, gammaCAC1, gammaCAC2, GATA-1, GATA-2, GATA-3, GATA-4, GATA-5, GATA-6, Gbx-1, Gbx-2, GCF, GCMa, GCNS, GF1, GLI, GLI3, GR alpha, GR beta, GRF-1, Gsc, Gscl, GT-IC, GT-IIA, GT-IIBalpha, GT-IIBbeta, H1TF1, H1TF2, H2RIIBP, H4TF-1, H4TF-2, HAND1, HAND2, HB9, HDAC1, HDAC2, HDAC3, hDaxx, heat-induced factor, HEB, HEB1-p67, HEB1-p94, HEF-1 B, HEF-1T, HEF-4C, HEN1, HEN2, Hesxl, Hex, HIF-1, HIF-lalpha, HIF-lbeta, HiNF-A, HiNF-B, HINF-C, HINF-D, HiNF-D3, HiNF-E, HiNF-P, HIFI, HIV-EP2, Hlf, HLTF, HLTF (Met123), HLX, HMBP, HMG I, HMG I(Y), HMG Y, HMGI-C, HNF-IA, HNF-IB, HNF-IC, HNF-3, HNF-3alpha, HNF-3beta, HNF-3gamma, HNF4, HNF-4alpha, HNF4alphal, HNF-4alpha2, HNF-4alpha3, HNF-4alpha4, HNF4gamma, HNF-6alpha, hnRNP K, HOX11, HOXAL HOXAIO, HOXAIO PL2, HOXA11, HOXA13, HOXA2, HOXA3, HOXA4, HOXAS, HOXA6, HOXA7, HOXA9A, HOXA9B, HOXB-1, HOXB13, HOXB2, HOXB3, HOXB4, HOXBS, HOXB6, HOXAS, HOXB7, HOXB8, HOXB9, HOXC10, HOXC11, HOXC12, HOXC13, HOXC4, HOXCS, HOXC6, HOXC8, HOXC9, HOXD10, HOXD11, HOXD12, HOXD13, HOXD3, HOXD4, HOXD8, HOXD9, Hp55, Hp65, HPX42B, HrpF, HSF, HSF1 (long), HSF1 (short), HSF2, hsp56, Hsp90, IBP-1, ICER-II, ICER-ligamma, ICSBP, Idl, Idl H′, Id2, Id3, Id3/Heir-1, IF1, IgPE-1, IgPE-2, IgPE-3, IkappaB, IkappaB-alpha, IkappaB-beta, IkappaBR, II-1 RF, IL-6 RE-BP, 11-6 RF, INSAF, IPF1, IRF-1, IRF-2, B, IRX2a, Irx-3, Irx-4, ISGF-1, ISGF-3, ISGF3alpha, ISGF-3gamma, 1st-1 , ITF, ITF-1, ITF-2, JRF, Jun, JunB, JunD, kappay factor, KBP-1, KER1, KER-1, Koxl, KRF-1, Ku autoantigen, KUP, LBP-1, LBP-la, LBX1, LCR-Fl, LEF-1, LEF-IB, LF-Al, LHX1, LHX2, LHX3a, LHX3b, LHXS, LHX6.1a, LHX6.1b, LIT-1, Lmol, Lmo2, LMX1A, LMX1B, L-Myl (long form), L-Myl (short form), L-My2, LSF, LXRalpha, LyF-1, Ly1-1, M factor, Madl, MASH-1, Maxl, Max2, MAZ, MAZ1, MB67, MBF1, MBF2, MBF3, MBP-1 (1), MBP-1 (2), MBP-2, MDBP, MEF-2, MEF-2B, MEF-2C (433 AA form), MEF-2C (465 AA form), MEF-2C (473 M form), MEF-2C/delta32 (441 AA form), MEF-2D00, MEF-2DOB, MEF-2DAO, MEF-2DAO, MEF-2DAB, MEF-2DA′B, Meis-1, Meis-2a, Meis-2b, Meis-2c, Meis-2d, Meis-2e, Meis3, Meoxl, Meoxla, Meox2, MHox (K-2), Mi, MIF-1, Miz-1, MM-1, MOP3, MR, Msx-1, Msx-2, MTB-Zf, MTF-1, mtTFl, Mxil, Myb, Myc, Myc 1, Myf-3, Myf-4, Myf-5, Myf-6, MyoD, MZF-1, NCI, NC2, NCX, NELF, NER1, Net, NF Ill-a, NF NF-1, NF-1A, NF-1B, NF-1X, NF-4FA, NF-4FB, NF-4FC, NF-A, NF-AB, NFAT-1, NF-AT3, NF-Atc, NF-Atp, NF-Atx, Nf etaA, NF-CLEOa, NF-CLEOb, NFdeltaE3A, NFdeltaE3B, NFdeltaE3C, NFdeltaE4A, NFdeltaE4B, NFdeltaE4C, Nfe, NF-E, NF-E2, NF-E2 p45, NF-E3, NFE-6, NF-Gma, NF-GMb, NF-IL-2A, NF-IL-2B, NF-jun, NF-kappaB, NF-kappaB(-like), NF-kappaBl, NF-kappaB 1, precursor, NF-kappaB2, NF-kappaB2 (p49), NF-kappaB2 precursor, NF-kappaEl, NF-kappaE2, NF-kappaE3, NF-MHCIIA, NF-MHCIIB, NF-muEl, NF-muE2, NF-muE3, NF-S, NF-X, NF-Xl, NF-X2, NF-X3, NF-Xc, NF-YA, NF-Zc, NF-Zz, NHP-1, NHP-2, NHP3, NHP4, NKX2-5, NKX2B, NKX2C, NKX2G, NKX3A, NKX3A vl, NKX3A v2, NKX3A v3, NKX3A v4, NKX3B, NKX6A, Nmi, N-Myc, N-Oct-2alpha, N-Oct-2beta, N-Oct-3, N-Oct-4, N-Oct-5a, N-Oct-Sb, NP-TCII, NR2E3, NR4A2, Nrfl, Nrf-1, Nrf2, NRF-2betal, NRF-2gammal, NRL, NRSF form 1, NRSF form 2, NTF, 02, OCA-B, Oct-1, Oct-2, Oct-2.1, Oct-2B, Oct-2C, Oct-4A, Oct4B, Oct-5, Oct-6, Octa-factor, octamer-binding factor, oct-B2, oct-B3, Otxl, Otx2, OZF, p107, p130, p28 modulator, p300, p38erg, p45, p49erg,-p53, p55, p55erg, p65delta, p67, Pax-1, Pax-2, Pax-3, Pax-3A, Pax-3B, Pax-4, Pax-5, Pax-6, Pax-6/Pd-5a, Pax-7, Pax-8, Pax-8a, Pax-8b, Pax-8c, Pax-8d, Pax-8e, Pax-8f, Pax-9, Pbx-la, Pbx-lb, Pbx-2, Pbx-3a, Pbx-3b, PC2, PC4, PCS, PEA3, PEBP2alpha, PEBP2beta, Pit-1, PITX1, PITX2, PITX3, PKNOX1, PLZF, PO-B, Pontin52, PPARalpha, PPARbeta, PPARgammal, PPARgamma2, PPUR, PR, PR A, pRb, PRD1-BF1, PRDI-BFc, Prop-1, PSE1, P-TEFb, PTF, PTFalpha, PTFbeta, PTFdelta, PTFgamma, Pu box binding factor, Pu box binding factor (B JA-B), PU.1 , PuF, Pur factor, R1 , R2, RAR-alphal, RAR-beta, RAR-beta2, RAR-gamma, RAR-gammal, RBP60, RBP-Jkappa, Rel, RelA, RelB, RFX, RFX1, RFX2, RFX3, RFXS, RF-Y, RORalphal, RORalpha2, RORalpha3, RORbeta, RORgamma, Rox, RPF1, RPGalpha, RREB-1, RSRFC4, RSRFC9, RVF, RXR-alpha, RXR-beta, SAP-la, SAP1b, SF-1, SHOX2a, SHOX2b, SHOXa, SHOXb, SHP, SIII-p110, SIII-p15, SIII-p18, SIM', Six-1, Six-2, Six-3, Six-4, Six-5, Six-6, SMAD-1, SMAD-2, SMAD-3, SMAD-4, SMAD-5, SOX-11, SOX-12, Sox-4, Sox-5, SOX-9, Spl, Sp2, Sp3, Sp4, Sph factor, Spi-B, SPIN, SRCAP, SREBP-la, SREBP-lb, SREBP-lc, SREBP-2, SRE-ZBP, SRF, SRY, SRPL Staf-50, STATlalpha, STATlbeta, STAT2, STAT3, STAT4, STAT6, T3R, T3R-alphal, T3R-alpha2, T3R-beta, TAF(I)110, TAF(I)48, TAF(I)63, TAF(II)100, TAF(II)125, TAF(II)135, TAF(II)170, TAF(II)18, TAF(II)20, TAF(II)250, TAF(II)250Delta, TAF(II)28, TAF(II)30, TAF(II)31, TAF(II)55, TAF(II)70-alpha, TAF(II)70-beta, TAF(II)70-gamma, TAF-I, TAF-II, TAF-L, Tal-1, Tal-lbeta, Tat-2, TAR factor, TBP, TBX1A, TBX1B, TBX2, TBX4, TBXS (long isoform), TBXS (short isoform), TCF, TCF-1, TCF-1A, TCF-1B, TCF-1C, TCF-1D, TCF-1E, TCF-1F, TCF-1G, TCF-2alpha, TCF-3, TCF-4, TCF-4(K), TCF-4B, TCF-4E, TCFbetal, TEF-1, TEF-2, tel, TFE3, TFEB, TFIIA, TFIIA-alpha/beta precursor, TFIIA-alpha/beta precursor, TFIIA-gamma, TFIIB, TFIID, TFIIE, TFIIE-alpha, TFIIE-beta, TFIIF, TFIIF-alpha, TFIIF-beta, TFIIH, TFIIH*, TFIIH-CAK, TFIIH-cyclin H, TFIIH-ERCC2/CAK, TFIIH-MAT1, TFIIH-M015, TFIIH-p34, TFIIH-p44, TFIIH-p62, TFIIH-p80, TFIIH-p90, TFII-I, Tf-LF1, Tf-LF2, TGIF, TGIF2, TGT3, THRAL TIF2, TLE1, TLX3, TMF, TR2, TR2-11, TR2-9, TR3, TR4, TRAP, TREB-1, TREB-2, TREB-3, TREFL TREF2, TRF (2), TTF-1, TXRE BP, TxREF, UBF, UBP-1, UEF-1, UEF-2, UEF-3, UEF-4, USF1, USF2, USF2b, Vav, Vax-2, VDR, vHNF-1A, vHNF-1B, vHNF-1C, VITF, WSTF, WT1, WT1I, WT1 I-KTS, WT1 I-de12, WT1-KTS, WT1-de12, X2BP, XBP-1, XW-V, XX, YAF2, YB-1, YEBP, YY1, ZEB, ZFl, ZF2, ZFX, ZHX1, ZIC2, ZID, ZNF 174, amongst others.

Disclosed herein are methods for analyzing the pattern of an epigenetic chromatin modification in a single cell or populations of cells. In some embodiments, the epigenetic chromatin modification is a histone modification or a DNA modification. Histone modifications targeted by the methods disclosed herein include but are not limited to H2A.X., H2A.Z, H2A.Zac, H2A.ZK4ac, H2A.ZK7ac, MAK 19ub, H2AK5ac, H2BK12ac, H2BK15ac, H2BK2Oac, H2BK123uh, H2Bpan, H3.3, H3K14ac, H3K48ac, H3K18mel, H3K18rne2, H3K23me2, H3K27ac, H3K27me1, H3K27me2, H3K27me3, H3K27me3S28p, H310611101, H3K36me2, U3K36tne3, H3K4ac, H3K4me1, H3K4me2, H3K4me3, H3K4me3T6p, H3k4un, H3K.56ac, H3K56mel, H3K64m03, H3K79ac, H3K79me1, H3K79me3, H3K9/14ac, H3K9ac, H3K9acS10p, H3K9me1, H3K9me2, H3K9me3, H3Kme3SlOp, H3K9un, H3pan, H3R17me2, H3R17me2(asym), H3R171ne2(asyin)KI8ac, H3R2rne2K4me2,113T6pK9me3, II4K1.2ac, H4K 16ac, H4K2Oac, H4K2Ornel, H4K2Oine2, H3R2me2, H4K2Ome3, H4K5,8,12ac, H4K5ac, H4K8ac, H4pan. and H4S1p.

Other non-limiting examples of chromatin-associated proteins that can be targeted using the methods disclosed herein include HDAC1, HDAC2, ItiFialpha, HPI, JARID1C, MU⁻2a, KAP1, KAT2B, KDM6A, LSD-., 1\413D1, MBD1, MeCP2, MYH11, NCOR1, NE-E2, NF&B, NFYB, NRF 1, NRF2, OCT4, p300, p53, PARP1, PAX8, Pol 11, Poi II S2p, PPARCi, RbAp48, RBBP5, RFX-AP, RNF2, SAP3O, SIN3A, Ski3, Ski8, SMAD1, SMAD2, SMYD3, Suzl 2, TALL TARDBP, TRP, TFHF, THOC1, TIPS, TRRAP, Tyl, UHRF1, YY1, ZHX2. and ZNIYM3. AF9, ML1 -ETO, BRD4, C/EBP, CBFb, CBX.2, CBX8, CHD1, CHD7, CRISPRICas9, CTCF, CXXCI, DNMT3B, E2F6, ERR, RTO, ⁻FM2, FOXAI, FOXA2, FOXMl, FUBP1, GR, and GTF2E2.

In one embodiment, the methods disclosed herein comprises contacting a chromatin-associated protein or a chromatin modification with a specific binding agent that specifically recognizes the chromatin-associated protein or chromatin modification.

In one embodiment, the specific binding agent is an antibody or an antigen-binding fragment thereof. Polyclonal or monoclonal antibodies and fragments of monoclonal antibodies such as Fab, F(ab′)2 and FIT fragments, as well as any other agent capable of specifically binding to a chromatin-associated protein or chromatin modification may be produced. Optimally, antibodies raised against a chromatin-associated protein or chromatin modification specifically bind the chromatin-associated protein or chromatin modification of interest. That is, such antibodies would recognize and bind the chromatin-associated protein or chromatin modification and would not substantially recognize or bind to other chromatin-associated protein or chromatin modifications. The determination that an antibody specifically binds the target or internalizing receptor polypeptide of interest may be made by any one of a number of standard immunoassay methods; for instance, the Western blotting technique (Sambrook et al., 1989, Molecular Cloning: A Laboratory Manual, Cold Spring Harbor Laboratory, Cold Spring Harbor, N.Y.).

In some embodiments, the method disclosed herein comprises contacting an uncrosslinked permeabilized cell with the specific binding agent. In some embodiments, the method disclosed herein comprises contacting a crosslinked permeabilized cell with the specific binding agent. In some embodiments, the contacting is performed at a temperature of about 4 C. The use of intact cells or nuclei preserves the native chromatin structure, which otherwise might be altered by fragmentation and other processing steps.

In some embodiments, the cell and/or the nucleus of the cell is permeabilized by contacting the cell with an agent that permeabilizes the cells, such as with a detergent, for example Triton and/or NP-40 or another agent, such as digitonin.

In some embodiment, the cell is eukaryotic cell derived from, for example, yeast, an insect, a fungus, a bird, or a mammal. In some embodiments, the mammalian cell is of human, primate, hamster, rabbit, rodent, cow, pig, sheep, horse, goat, dog or cat origin, but any other mammalian cell may be used.

In some embodiments, the specific binding agent is linked to a transposase that is optionally inactive and activatable, for example by addition of an ion such as a cation such as Mg²⁺. Once activated, the transposase is able to excise the sequence of DNA bound to the chromatin-associated protein or chromatin modification.

In some embodiments, the transposase is a Tn5 transposase. In some embodiments, the transposase is a hyperactive Tn5 transposase. In some embodiments, the transposase is a MuA transposase. Additional, non-limiting examples of transposition systems that can be used with embodiments provided herein include Staphylococcus aureus Tn552 (Colegio et al, J. Bacteriol, 183: 2384-8, 2001 ; Kirby C et al, Mol. Microbiol, 43: 173-86, 2002), Tyl (Devine & Boeke, Nucleic Acids Res., 22: 3765-72, 1994 and International Publication WO 95/23875), Transposon Tn7 (Craig, N L, Science. 271 : 1512, 1996; Craig, N L, Review in: Curr Top Microbiol Immunol, 204:27-48, 1996), Tn/O and IS 10 (Kleckner N, et al, Curr Top Microbiol Immunol, 204:49-82, 1996), Mariner transposase (Lampe D J, et al, EMBO J., 15: 5470-9, 1996), Tel (Plasterk R H, Curr. Topics Microbiol. Immunol, 204: 125-43, 1996), P Element (Gloor, G B, Methods Mol. Biol, 260: 97-1 14, 2004), Tn3 (Ichikawa & Ohtsubo, J Biol. Chem. 265: 18829-32, 1990), bacterial insertion sequences (Ohtsubo & Sekine, Curr. Top. Microbiol. Immunol. 204: 1-26, 1996), retroviruses (Brown, et al, Proc Natl Acad Sci USA, 86:2525-9, 1989), and retrotransposon of yeast (Boeke & Corces, Annu Rev Microbiol. 43 :403-34, 1989). More examples include ISS, Tn10, Tn903, IS91 1, and engineered versions of transposase family enzymes (Zhang et al, (2009) PLoS Genet. 5:e1000689. Epub 2009 Oct 16; Wilson C. et al (2007) J. Microbiol. Methods 71 :332-5) and those described in U.S. Pat. Nos. 5,925,545; 5,965,443; 6,437,109; 6,159,736; 6,406,896; 7,083,980; 7,316,903; 7,608,434; 6,294,385; 7,067,644, 7,527,966; and International Patent Publication No. WO2012103545, all of which are specifically incorporated herein by reference in their entireties.

In some embodiments, the transposase is loaded with a nucleic acid comprising one or more tags. The tag may comprise a sequence that facilitates the sequencing of the fragmented DNA produced, for example using next generation sequencing, such as paired end, and/or array-based sequencing. The tag may comprise an endonuclease restriction site. The tag may comprise a barcode sequence for identification of a specific sample or replicate. As used herein, a barcode is an oligonucleotide (double or single stranded) with a specific sequence. The tag may comprise a linker sequence. The tag may comprise a universal priming site. The inclusion of a universal priming site facilitates the amplification of the fragmented DNA produced, for example using PCR based amplification. In one embodiment, the primer sequence can be complementary to a primer used for amplification. In one embodiment, the primer sequence is complementary to a primer used for sequencing. The tag may provide the nucleic acid with some functionality and may comprise an affinity or reporter moiety.

In some embodiments, the transposase is linked to a second binding agent that binds to the specific binding agent that specifically recognizes the chromatin-associated protein or chromatin modification.

In some embodiments, the specific binding agent that specifically recognizes the chromatin-associated protein or chromatin modification is an antibody. In some embodiments, the transposase is linked to a second antibody that binds to the first antibody that specifically recognizes the chromatin-associated protein or chromatin modification. In some embodiments, the transposase is linked to protein A or protein G that binds to the first antibody that specifically recognizes the chromatin-associated protein or chromatin modification. The transposase may be fused to all or part of the staphylococcal protein A (pA) or to all or part of staphylococcal protein G (pG) or to both pA and pG (pAG). The transposase may also be fused to any other protein or protein moiety, for example derivatives of pA or pG, which has an affinity for antibodies. In one embodiment, the transposase is fused to pAG-MN. In pAG-MN, the pA moiety contains 2 IgG binding domains of staphylococcal protein A, i.e., amino acids 186 to 327 of (Genbank entry AAA26676; protein A from Staphylococcus aureus) (SEQ ID NO:1). Variants that retain the activity are also contemplated, such as those having a sequence identity of at least 70%, at least 80%, at least 90%, at least 95%, at least 96%, at least 97%, at least 98%, or at least 99% identity to amino acids 186 to 327 of Genbank entry AAA26676. SEQ ID NO:1 (corresponds to amino acids 186 to 327 of Genbank entry AAA26676:

SLKDDPSQSANLLSEAKKLNESQAPKADNKFNKEQQNAFYEILHLPNLN
EEQRNGFIQSLKDDPSQSANLLAEAKKLNDAQAPKADNKFNKEQQNAFY
EILHLPNLTEEQRNGFIQSLKDDPSVSKEILAEAKKLNDAQAPK

Provided herein is a method comprising contacting a nucleus with a first antibody that specifically binds to a chromatin-associated protein or chromatin modification and contacting the nucleus with a transposase linked to a second antibody that binds to the first antibody. Provided herein is a method comprising contacting a nucleus with a first antibody that specifically binds to a chromatin-associated protein or chromatin modification and contacting the nucleus with a transposase linked to protein A or protein G that binds to the first antibody.

In some embodiments, the specific binding agent and the transposase are pre-incubated with each other before the cells are contacted with the binding agent/transposase complex. In some embodiments, the specific binding agent that binds to a chromatin-associated factor or chromatin modification is an antibody, wherein the antibody is pre-incubated with a transposase linked to a binding moiety that binds to the antibody; and subsequently one or more nuclei are contacted with the antibody bound to the transposase.

Provided herein is a method comprising contacting a nucleus with a first antibody that specifically binds to a chromatin-associated protein or chromatin modification, contacting the nucleus with second antibody that binds to the first antibody, and contacting the nucleus with a transposase linked to a third antibody that binds to the first antibody.

In some embodiments, the nucleus is contacted with more than one transposase.

In one aspect, provided is a method comprising:

(1) permeabilizing one or more nuclei;

(2) (i) contacting the one or more nuclei with an antibody that binds to a chromatin-associated protein or chromatin modification; and contacting the one or more nuclei with a transposase linked to a binding moiety that binds to the antibody; (ii) incubating the antibody that binds to a chromatin-associated protein or chromatin modification with the transposase linked to a binding moiety that binds to the antibody; and contacting the one or more nuclei with the antibody bound to the transposase; or (iii) contacting the one or more nuclei with an antibody that binds to a chromatin-associated protein or chromatin modification, wherein the antibody is covalently linked to a transposase;

wherein the transposase is loaded with a nucleic acid comprising a tag; and

(3) initiating a tagmentation reaction, resulting in the generation of genomic DNA fragments comprising the tag.

In some embodiments, the one or more nuclei are contacted with more than one antibody that binds to a chromatin-associated protein or chromatin modification. In some embodiments, the transposase is loaded with a nucleic acid comprising a tag, wherein the tag comprises a nucleic acid comprising a barcode and/or an endonuclease restriction site. In some embodiments, the one or more nuclei are contacted with more than one transposase. In some embodiments, the one or more nuclei are contacted with one or more transposases, wherein each transposase is loaded with a nucleic acid comprising a different tag. In some embodiments, the binding moiety linked to the transposase is protein A.

Reverse Transcription

In one aspect, provided is a method comprising:

(1) permeabilizing one or more nuclei;

(2) reverse transcribing the RNA in the one or more nuclei using primers comprising a tag, resulting in the generation of cDNA comprising the tag.

In some embodiments, the tag comprises a barcode and/or an endonuclease restriction site tag. In some embodiments, the tag comprises a sequence that facilitates the sequencing of the fragmented DNA produced, a linker sequence, a universal priming site or another moiety that equips the reverse transcription product with some functionality such as an affinity tag or a reporter moiety.

Any enzyme suitable for reverse transcription can be used.

In one aspect, provided is a method comprising:

(1) permeabilizing one or more nuclei;

(2) (i) contacting the one or more nuclei with an antibody that binds to a chromatin-associated protein or chromatin modification; and contacting the one or more nuclei with a transposase linked to a binding moiety that binds to the antibody; (ii) incubating the antibody that binds to a chromatin-associated protein or chromatin modification with the transposase linked to a binding moiety that binds to the antibody; and contacting the one or more nuclei with the antibody bound to the transposase; or (iii) contacting the one or more nuclei with an antibody that binds to a chromatin-associated protein or chromatin modification, wherein the antibody is covalently linked to a transposase;

wherein the transposase is loaded with a nucleic acid comprising a first tag; and

(3) initiating a tagmentation reaction, resulting in the generation of genomic DNA fragments comprising the first tag; and

(4) reverse transcribing the RNA in the one or more nuclei using primers comprising a second tag, resulting in the generation of cDNA comprising the second tag.

In some embodiments, the one or more nuclei are contacted with more than one antibody that binds to a chromatin-associated protein or chromatin modification. In one embodiment, the first and the second tag comprise the same barcode. In one embodiment, the first tag comprises a first endonuclease restriction site and the second tag comprises a second endonuclease restriction site. In one embodiment, the first and the second tag comprise the same barcode, the first tag comprises a first endonuclease restriction site, and the second tag comprises a second endonuclease restriction site. In some embodiments, the binding moiety linked to the transposase is protein A. In one embodiment, the tagmentation reaction is carried out before the reverse transcription reaction. In one embodiment, the tagmentation reaction is carried out after the reverse transcription reaction. In one embodiment, the tagmentation reaction and the reverse transcription reaction are carried our simultaneously.

In one embodiment, provided is a method comprising:

(1) permeabilizing one or more nuclei;

(2) (i) contacting the one or more nuclei with an antibody that binds to a chromatin-associated protein or chromatin modification; and contacting the one or more nuclei with a transposase linked to a protein A; (ii) incubating the antibody that binds to a chromatin-associated factor or chromatin modification with the transposase linked to a protein A; and contacting the one or more nuclei with the antibody bound to the transposase; or (iii) contacting the one or more nuclei with an antibody that binds to a chromatin-associated protein or chromatin modification, wherein the antibody is covalently linked to a transposase;

wherein the transposase is loaded with a nucleic acid comprising a first tag comprising a barcode and a first restriction site; and

(3) initiating a tagmentation reaction, resulting in the generation of genomic DNA fragments comprising the first tag; and

(4) reverse transcribing the RNA in the one or more nuclei using primers comprising a second tag comprising the barcode and a second restriction site, resulting in the generation of cDNA comprising the second tag.

Provided is a method comprising providing a sample comprising nuclei and dividing the sample into two or more sub-samples, and for each of the two or more sub-samples, performing a method comprising:

(1) permeabilizing the nuclei;

(2) (i) contacting the nuclei with an antibody that binds to a chromatin-associated protein or chromatin modification; and contacting the nuclei with a transposase linked to a binding moiety that binds to the antibody; (ii) incubating the antibody that binds to a chromatin-associated protein or chromatin modification with the transposase linked to a binding moiety that binds to the antibody; and contacting the one or more nuclei with the antibody bound to the transposase; or (iii) contacting the one or more nuclei with an antibody that binds to a chromatin-associated protein or chromatin modification, wherein the antibody is covalently linked to a transposase;

wherein the transposase is loaded with a nucleic acid comprising a first tag comprising a barcode; and

(3) initiating a tagmentation reaction, resulting in the generation of genomic DNA fragments comprising the first tag; and

(4) reverse transcribing the RNA in the nuclei using primers comprising a second tag comprising the barcode of the first tag, resulting in the generation of cDNA comprising the second tag.

Ligation-Based Combinatorial Barcoding

In embodiments, the nuclei comprising genomic DNA fragments comprising a first tag and the cDNA comprising a second tag are subjected to additional barcoding. In some embodiments, a third tag is ligated to the genomic DNA fragments comprising a first tag and to the cDNA comprising a second tag. In some embodiments, the third tag comprises a barcode and/or an endonuclease restriction site. In some embodiments, a fourth tag is ligated to the genomic DNA fragments comprising a first tag and a third tag and to the cDNA comprising a second tag and a third tag. In some embodiments, the fourth tag adaptor comprises a barcode and/or an endonuclease restriction site. Additional tags may be ligated to the resulting genomic DNA fragments comprising a first, third, and fourth tag and to the cDNA comprising a second, third, and fourth tag.

In one aspect, provided is a method comprising:

(1) providing nuclei comprising genomic DNA fragments comprising a first tag comprising a barcode and cDNA comprising a second tag comprising the barcode of the first tag;

(2) contacting the nuclei with a ligase and a third tag comprising a second barcode, resulting in the generation of genomic DNA fragments comprising a first tag and a third tag and cDNA comprising a second tag and a third tag; and optionally

(3) repeating step 2 once or multiple times to add additional tags the genomic DNA and the cDNA.

Provided is a method comprising providing a sample comprising nuclei and dividing the sample into two or more sub-samples, wherein each sub-sample is subjected to tagmentation and reverse transcription, and wherein the resulting genomic DNA and the cDNA of each sub-sample in the nuclei of each sub-sample incorporate the same barcode selected from a first set of barcodes, but wherein the barcodes used for the different sub-samples are different (first round of barcoding). The different sub-samples may then be pooled and divided again into two or more sub-samples, wherein each of the two or more sub-samples is contacted with a ligase and an adaptor comprising a barcode selected form a second set of barcodes to ligate the adaptor to the genomic DNA and the cDNA in each sub-sample (second round of barcoding). The different sub-samples may then be again pooled and divided again into two or more sub-samples, wherein each of the two or more sub-samples is contacted with a ligase and an adaptor comprising a different barcode selected from a third set of barcodes to ligate the adaptor to the genomic DNA and the cDNA in each sub-sample (third round of barcoding). This process can be repeated to allow for additional rounds of barcoding.

Provided is a method comprising:

(1) providing a sample comprising nuclei;

(2) dividing the sample into a first set of sub-samples comprising two or more sub-samples;

(3) permeabilizing the nuclei in the two or more sub-samples in the first set of sub-samples;

(4) (i) contacting the nuclei in the two or more sub-samples in the first set of sub-samples with an antibody that binds to a chromatin-associated protein or chromatin modification; and contacting each of the two or more sub-samples in the first set of sub-samples with a transposase linked to a binding moiety that binds to the antibody; (ii) incubating the antibody that binds to a chromatin-associated protein or chromatin modification with the transposase linked to a binding moiety that binds to the antibody; and contacting the one or more nuclei with the antibody bound to the transposase; or (iii) contacting the one or more nuclei with an antibody that binds to a chromatin-associated protein or chromatin modification, wherein the antibody is covalently linked to a transposase;

wherein the transposase is loaded with a nucleic acid comprising a first tag comprising a barcode selected from a first set of barcodes;

(5) initiating a tagmentation reaction, resulting in the generation of genomic DNA fragments comprising the first tag;

(6) reverse transcribing the RNA in nuclei using primers comprising a second tag comprising the barcode of the first tag, resulting in the generation of cDNA comprising the second tag;

(7) pooling the first set of sub-samples to generate a first sub-sample pool;

(8) dividing the first sub-sample pool into two or more sub-samples to generate a second set of sub-samples;

(9) contacting each of the two or more sub-samples in the second set of sub-samples with a ligase and a tag comprising a barcode selected from a second set of barcodes, wherein the tag is ligated to the genomic DNA and the cDNA;

(10) pooling the second set of sub-samples to generate a second sub-sample pool;

(11) dividing the second sub-sample pool into two or more sub-samples to generate a third set of sub-samples;

(12) contacting each of the two or more sub-samples in the third set of sub-samples with a ligase and a tag comprising a barcode selected from a third set of barcodes, wherein the tag is ligated to the genomic DNA and the cDNA;

(13) optionally repeating steps (10)-(12) with a fourth set of barcodes.

In some embodiments, the steps of pooling sub-samples, dividing into new sub-samples, and contacting the new sub-samples with a ligase and a tag comprising an additional barcode are repeated on or more times.

Lysis of Nuclei

In some embodiments, after the genomic DNA and the cDNA (obtained by reverse transcription of RNA) contained in a nucleus has undergone one or more rounds of barcoding, the nucleus is lysed, releasing the DNA and cDNA. The DNA and cDNA of multiple cells can be pooled to generate a DNA/cDNA pool.

Preamplification of Barcoded DNA/cDNA

In some embodiments, the DNA and cDNA in the DNA/cDNA pool is subjected to polynucleotide tailing with terminal deoxynucleotidyltransferase (TdT), resulting in the addition of a homopolymeric sequence at its 3′-end that can then be used as an anchor for amplification.

In one embodiment, the DNA and cDNA in the DNA/cDNA pool is subjected to polynucleotide tailing by contacting the DNA and cDNA with a DNA ligase and DNA or RNA oligonucleotide. In some embodiments, the DNA ligase is a T3, T4 or T7 DNA ligase. In one embodiment, the DNA and cDNA in the DNA/cDNA pool is subjected to polynucleotide tailing by contacting the DNA and cDNA with a DNA polymerase and a random primer. In one embodiment, the DNA and cDNA in the DNA/cDNA pool is subjected to polynucleotide tailing by contacting the DNA and cDNA with a DNA or RNA oligonucleotide with reactive chemical group that attaches to the 3′-end of the DNA and cDNA. In some embodiments, the reactive chemical group is an azide group or an alkyne group.

In some embodiments, the polynucleotide tailed DNA and cDNA are pre-amplified by PCR. In some embodiments, at least one of the primers used for the amplification of the polynucleotide tailed DNA comprises a restriction site for a type IIS endonuclease.

A type IIS restriction enzyme is an enzyme that recognizes asymmetric DNA sequences and cleaves at a defined distance outside of their recognition sequence, usually within 1 to 20 nucleotides. Examples of type IIS restriction enzymes compatible with the compositions and methods disclosed herein include, but are not limited to, FokI, AcuI, AsuHPI, BbvI, BpmI, BpuEI, BseMII, BseRI, BseXI, BsgI, BslFI, BsmFI, BsPCNI, BstV1I, BtgZI, EciI, Eco57I, FaqI, GsuI, HphI, MmeI, NmeAIII, SchI, TaqII, TspDTI, TspGWI.

Generation of Separate DNA and RNA Sequencing Libraries

In some embodiments, the pool comprising polynucleotide tailed DNA and cDNA is used to generate two separate libraries, a DNA and an RNA library. As used herein, the term “RNA library” refers to a library of cDNA molecules that have been prepared by reverse transcribing the RNA present in the nuclei (and optionally amplifying and further modifying the resulting cDNA).

Various methods can be used for generating a DNA and an RNA library from the pool comprising polynucleotide tailed DNA and cDNA.

In one aspect, provided is a method for generating a DNA and an RNA library from the pool comprising polynucleotide tailed DNA and cDNA, wherein the genomic DNA is linked to a tag comprising a first endonuclease restriction site and the cDNA is linked to a tag comprising a second endonuclease restriction site. The pool comprising the polynucleotide-tailed DNA and cDNA may be divided into two batches, wherein (i) the first batch is digested with a first endonuclease cleaving the amplified polynucleotide tailed DNA at the first endonuclease restriction site, generating an RNA library and (ii) the second batch is digested with a second endonuclease cleaving the amplified polynucleotide tailed cDNA at the second endonuclease restriction site, generating a DNA library.

In one aspect, provided is a method for generating a DNA and an RNA library from the pool comprising polynucleotide tailed DNA and cDNA, wherein the genomic DNA is linked to a tag comprising a first endonuclease restriction site and the cDNA is linked to a tag comprising a second endonuclease restriction site. The pool comprising the polynucleotide-tailed DNA and cDNA may be divided into two batches.

In one embodiment, the first batch is subjected to the following steps: (a) cleaving the amplified polynucleotide tailed DNA with a first restriction enzyme recognizing the first restriction site; and (b) contacting the amplified polynucleotide tailed cDNA with a second transposase loaded with a nucleic acid comprising a sequencing adaptor and initiating a tagmentation reaction, resulting in the generation of amplified polynucleotide tailed cDNA comprising the sequencing adaptor; generating an RNA library.

In one embodiment, one of the primers used for the amplification of the genomic DNA comprises a restriction site for a third endonuclease, thus introducing a third restriction site into the amplified polynucleotide tailed DNA. In one embodiment, the second batch is subjected to the following steps: (a) cleaving the amplified polynucleotide tailed cDNA with a second endonuclease cleaving at the second endonuclease restriction site; (b) cleaving the amplified polynucleotide tailed DNA with a third endonuclease that recognizes the third restriction site; and (c) contacting the DNA end with a sequencing adaptor and a ligase, resulting in the generation of amplified polynucleotide tailed DNA comprising the sequencing adaptor; generating a DNA library.

In one embodiment, one of the primers used for the amplification of the genomic DNA comprises a restriction site for a Type IIS endonuclease, thus introducing a third restriction site into the amplified polynucleotide tailed DNA. In one embodiment, the second batch is subjected to the following steps: (a) cleaving the amplified polynucleotide tailed cDNA with a second endonuclease cleaving at the second endonuclease restriction site; (b) cleaving the amplified polynucleotide tailed DNA with a restriction a Type IIS endonuclease that recognizes the third restriction site, wherein the Type IIS endonuclease generates a sticky DNA end; and (c) contacting the sticky DNA end with a sequencing adaptor and a ligase, resulting in the generation of amplified polynucleotide tailed DNA comprising the sequencing adaptor; generating a DNA library.

In one aspect, provided is a method for generating a DNA and an RNA library from the pool comprising polynucleotide tailed DNA and cDNA wherein the genomic DNA is linked to a tag comprising a first endonuclease restriction site and the cDNA is linked to a tag comprising a second endonuclease restriction site. The pool comprising the polynucleotide-tailed DNA and cDNA may be divided into two batches.

In one embodiment, one of the primers used for the amplification of the cDNA comprises a restriction site for a third endonuclease, thus introducing a third restriction site into the amplified polynucleotide tailed cDNA. In one embodiment, the first batch is subjected to the following steps: (a) cleaving the amplified polynucleotide tailed DNA with a first restriction enzyme recognizing the first restriction site; (b) cleaving the amplified polynucleotide tailed cDNA with a third endonuclease that recognizes the third restriction site; and (c) contacting the cDNA end with a sequencing adaptor and a ligase, resulting in the generation of amplified polynucleotide tailed cDNA comprising the sequencing adaptor; generating an RNA library.

In one embodiment, one of the primers used for the amplification of the cDNA comprises a restriction site for a Type IIS endonuclease, thus introducing a third restriction site into the amplified polynucleotide tailed cDNA. In one embodiment, the first batch is subjected to the following steps: (a) cleaving the amplified polynucleotide tailed DNA with a first restriction enzyme recognizing the first restriction site; (b) cleaving the amplified polynucleotide tailed cDNA with a restriction a Type IIS endonuclease that recognizes the third restriction site, generating, wherein the Type IIS endonuclease generates a sticky cDNA end; and (c) contacting the sticky cDNA end with a sequencing adaptor and a ligase, resulting in the generation of amplified polynucleotide tailed cDNA comprising the sequencing adaptor; generating a DNA library.

In one embodiment, the second batch is subjected to the following steps: (a) cleaving the amplified polynucleotide tailed cDNA with a second endonuclease cleaving at the second endonuclease restriction site; and (b) contacting the amplified polynucleotide tailed DNA with a second transposase loaded with a nucleic acid comprising a sequencing adaptor and initiating a tagmentation reaction, resulting in the generation of amplified polynucleotide tailed DNA comprising the sequencing adaptor; generating a DNA library.

In one aspect, provided is a method for generating a DNA and an RNA library from the pool comprising polynucleotide tailed DNA and cDNA using click chemistry. As used herein, click chemistry refers to a class of biocompatible small molecule reactions commonly used in bioconjugation, allowing the joining of substrates of choice with specific biomolecules.

In some embodiments, the method comprises
a. contacting the one or more nuclei with an antibody that binds to a chromatin-associated protein or chromatin modification; and a first transposase; wherein the first transposase is loaded with a nucleic acid comprising a first tag, wherein the first tag comprises a first barcode selected from a first set of barcodes;
b. initiating a tagmentation reaction, resulting in the generation of genomic DNA fragments comprising the first tag;
c. reverse transcribing the RNA in the one or more nuclei using primers comprising a second tag, wherein the second tag comprises the barcode of the first tag, resulting in the generation of cDNA comprising the second tag;
   wherein the first tag further comprises (i) a first reactive group suitable to perform click chemistry or (ii) a first affinity tag and/or wherein the second tag further comprises (i) a second reactive group suitable to perform click chemistry or (ii) a second affinity tag;
d. contacting the one or more nuclei with a ligase and a third tag comprising a second barcode selected from a second set of barcodes, resulting in the generation of genomic DNA fragments comprising a first tag and a third tag and cDNA comprising a second tag and a third tag;
e. lysing the one or more nuclei;
f. (I) contacting the genomic DNA fragments with an immobilized agent that
   (i) reacts with the first reactive group; or
   (ii) binds to the first affinity tag; and
   performing a pull-down of the genomic DNA to separate the genomic DNA from the cDNA; and/or
(II) contacting the cDNA with an immobilized agent that
   (i) reacts with the second reactive group; or
   (ii) binds to the second affinity tag; and performing a pull-down of the cDNA to separate the genomic cDNA from the DNA;
g. for the DNA library: contacting the genomic DNA with random primers comprising a sequencing adaptor, generating polynucleotide tailed DNA; and amplifying the polynucleotide tailed DNA;
h. for the RNA library: contacting the immobilized cDNA with random primers comprising a sequencing adaptor, generating polynucleotide tailed cDNA; and amplifying the polynucleotide tailed cDNA;
i. sequencing the molecules in the RNA library and the DNA library;
j. correlating the RNA library and the DNA library for each of the one or more nuclei.

In one embodiment, only the DNA is labeled with a reactive group suitable to perform click chemistry or (ii) an affinity tag. In one embodiment, only the cDNA is labeled with a reactive group suitable to perform click chemistry or (ii) an affinity tag. In some embodiments, both the DNA and the cDNA are labeled with (i) a reactive group suitable to perform click chemistry or (ii) an affinity tag, wherein the DNA and the cDNA are not labeled with the same reactive group suitable to perform click chemistry or affinity tag.

In some embodiments, the DNA is labeled with an affinity tag and the cDNA is labeled with a reactive group suitable to perform click chemistry. In some embodiments, the cDNA is labeled with an affinity tag and the DNA is labeled with a reactive group suitable to perform click chemistry. In some embodiments, the DNA or the cDNA is labeled with biotin, and the immobilized agent that binds to biotin is streptavidin. In some embodiments, the DNA or the cDNA is labeled with azide, and the immobilized agent that reacts with azide is DBCO.

Pairs of affinity tag/immobilized binding agent other than biotin/streptavidin may be used. Click chemistry pairs other than azide/DBCO may be used.

A person skilled in the art may identify variations of the methods described above. For instance, in some embodiments, the DNA molecules are labeled, for example using using biotin- or azide Tn5 adaptors. The pull-down of the labeled DNA may be followed by library preparation and sequencing. The cDNA molecules remaining in the supernatant can likewise be used for library preparation and sequencing as well.

In some embodiments, the cDNA molecules are labeled, for example using biotin- or azide labeled reverse transcription primers. The pull-down of the labeled cDNA may be followed by library preparation and sequencing. The DNA molecules remaining in the supernatant can likewise be used for library preparation and sequencing as well.

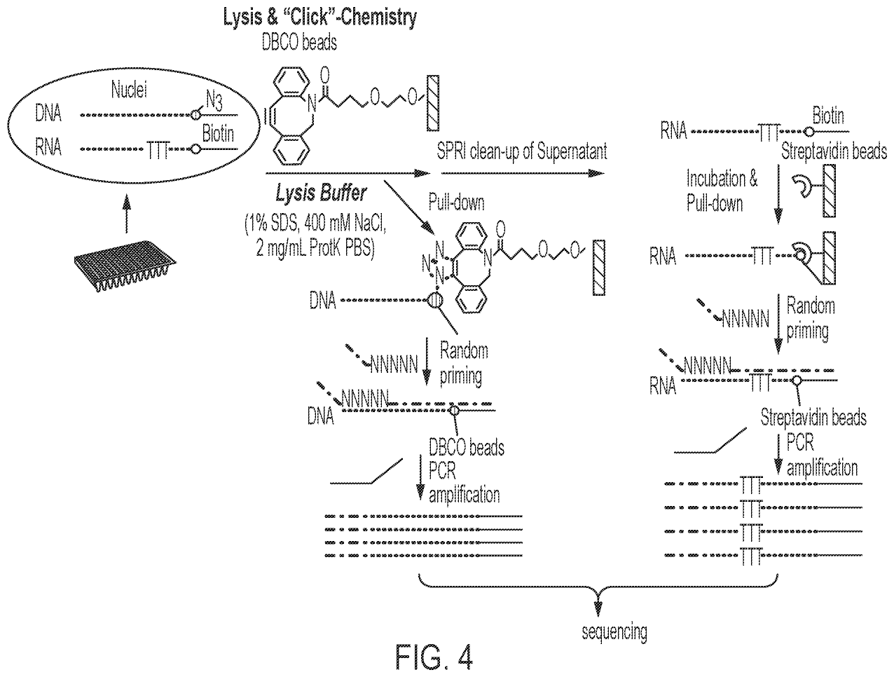

Non-limiting examples for methods of separating DNA and RNA libraries are shown in FIG. 4.

High Throughput Methods

In certain embodiments, the disclosed methods are provided that allow sample processing in a high-throughput manner. For example, 2, 3, 4, 5, 6, 7, 8, 9, 10, 50, 100, 200, 500, 750, 1000, or more chromatin-associated proteins and/or chromatin modifications may be analyzed in parallel. In one embodiment, up to 96 samples may be processed at once, using e.g., a 96-well plate. In other embodiments, fewer or more samples may be processed, using e.g., 6-well, 12-well, 32-well, 384-well or 1536-well plates. In some embodiments, the methods provided can be carried out in tubes, such as, for example, common 0.5 ml, 1.5 ml or 2.0 ml size tubes. These tubes may be arrayed in tube racks, floats or other holding devices.

The methods of the disclosure are useful for the joint analysis of regulation of gene expression and gene expression in a single cell or populations of cells. In a preferred embodiment, the methods are used for the joint analysis of regulation of gene expression and gene expression on a single cell level.

Applications

The methods disclosed herein are useful for analyzing the epigenome for different cell types, which is crucial for delineating the gene regulatory programs in different cell lineages during development and in pathological conditions. Further, by simultaneously assessing the transcriptional profiles along with chromatin states from the same cells, the methods disclosed herein provide a better understanding of gene regulatory mechanisms. For example, the methods disclosed herein are useful for identifying distinct groups of genes subject to divergent epigenetic regulatory mechanisms in different cell types and provide insights into the gene regulatory processes in different tissues. The methods disclosed herein are also useful for the genome-wide profiling of histone modifications, which can reveal not only the location and activity state of transcriptional regulatory elements, but also the regulatory mechanisms involved in cell-type-specific gene expression during development and disease pathology.

Through the joint analysis of regulation of gene expression and gene expression, the methods disclosed herein are useful for providing a “gene regulation/gene expression profile” that provides information about, for example, the interactions of a target nucleic acid with a chromatin-associated protein and/or certain histone/DNA modifications as well as the associated gene expression profile. The gene regulation/gene expression profile is particularly suited to diagnosing and/or monitoring disease states, such as disease state in an organism, for example a plant or an animal subject, such as a mammalian subject, for example a human subject. Certain disease states may be caused and/or characterized differential binding or proteins and/or nucleic acids to chromatin DNA in vivo. For example, certain interactions may occur in a diseased cell but not in a normal cell. In other examples, certain interactions may occur in a normal cell but not in diseased cell. Accordingly, provided are methods for correlating a gene regulation/gene expression profile with a disease state, for example cancer, or an infection, such as a viral or bacterial infection. It is understood that a correlation to a disease state could be made for any organism, including without limitation plants, and animals, such as humans. The gene regulation/gene expression profile correlated with a disease can be used as a “fingerprint” to identify and/or diagnose a disease in a cell, by virtue of having a similar “fingerprint.” The gene regulation/gene expression profile can be used to identify binding proteins and/or nucleic acids that are relevant in a disease state such as cancer, for example to identify particular proteins and/or nucleic acids as potential diagnostic and/or therapeutic targets. In addition, gene regulation/gene expression profile can be used to monitor a disease state, for example to monitor the response to a therapy, disease progression and/or make treatment decisions for subjects.

The ability to obtain a gene regulation/gene expression profile allows for the diagnosis of a disease state, for example by comparison of the gene regulation/gene expression profile present in a sample with the correlated with a specific disease state, wherein a similarity in profile indicates a particular disease state. Accordingly, provided herein are methods for diagnosing a disease state based on a gene regulation/gene expression profile correlated with a disease state, for example cancer, or an infection, such as a viral or bacterial infection. It is understood that a diagnosis of a disease state could be made for any organism, including without limitation plants, and animals, such as humans.

Also provided herein are methods for the correlation of an environmental stress or state with a gene regulation/gene expression profile, for example a whole organism, or a sample, such as a sample of cells, for example a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value.

Also provided herein are methods for screening libraries for agents that modulate interaction profiles, for example that alter the gene regulation/gene expression profile from an abnormal one, for example correlated to a disease state to one indicative of a disease free state. By exposing cells, tissues, or even whole animals, to different members of the chemical libraries, and performing the methods described herein, different members of a chemical library can be screened for their effect on interaction profiles simultaneously in a relatively short amount of time, for example using a high throughput method.

It is to be understood that this invention is not limited to the particular methodologies, or protocols described, as these may vary. Any methods and materials similar or equivalent to those described herein can be used in the practice or testing of embodiments of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

All referenced patents and applications are incorporated herein by reference in their entireties.

To facilitate a better understanding of the present invention, the following examples of specific embodiments are given. The following examples should not be read to limit or define the entire scope of the invention.

### EXAMPLES

### Example 1

Methods

Cell culture

HeLa S3 (human, ATCC CCL-2.2) cells were cultured according to standard procedures in Dulbecco's Modified Eagles' Medium supplemented with 10% fetal bovine serum (FBS) and 1% penicillin-streptomycin at 37° C. with 5% CO₂. Cells were not authenticated nor tested for mycoplasma. To prepare nuclei, HeLa S3 cells were harvested by centrifugation (300 g for 5 min), washed with PBS and counted using BioRad TC20 cell counter. The cells were then resuspended in cold Nuclei Permeabilization Buffer 1 (NPB1: 10 mM Tris-HCl pH 7.4, 10 mM NaCl, 3 mM MgCl₂, 1X Protease Inhibitor, 0.5 U/μL RNase OUT (ribonuclease inhibitor) and 0.5 U/μL SUPERase Inhibitor (RNase inhibitor) with 0.1% IGEPAL CA-630 (octylphenoxypolyethoxyethanol, a nonionic, non-denaturing detergent) and centrifuged for 10 min at 1,000 g, 4° C. and proceed to Paired-Tag experiments.

Processing of Biospecimens

Male C57BL/6J mice were purchased from Jackson laboratories at 8 weeks of age and maintained in the Salk animal barrier facility on 12-hr dark-light cycles with food ad libitum for four weeks before dissection. The frontal cortex and hippocampus were dissected and snap-frozen in dry ice. All protocols were approved by the Salk Institute's Institutional Animal Care and Use Committee (IACUC).

Single-cell suspension were prepared from douncing of the frozen tissues, in Doucing Buffer with Protease/RNase Inhibitor cocktail (DBI: 0.25 M sucrose, 25 mM KCl, 5 mM MgCl₂, 10 mM Tris-HCl pH 7.4, 1 mM DTT, 1X Protease Inhibitor, 0.5 U/μL RNase OUT and 0.5 U/μL SUPERase Inhibitor) supplemented with 0.1% Triton-X 100. For this, 10 nt 10% Triton-X100 was added into the douncer (1 mL), and 1 mL Douncing Buffer was added. The tissue dissection was transferred into the douncer. Loose pestle was used 5-10 times gently followed by tight pestle for 15-20 times. The cell suspension was then filtered by 30 μm Cell-Tric and spun-down for 10 min, 1,000 g at 4° C. After washing the cell pellets with DBI and spun-down again, NIB with 0.2% IGEPAL CA-630 was added to resuspend the nuclei pellets inl mL (5 million cells) and optionally rotated for 10 min at 4° C. The nuclei were counted by BioRad TC20 cell counter and proceed to Paired-Tag experiments immediately.

Annealing of Adaptors

To prepare the DNA barcoded plates (barcode rounds # 2 and # 3), 6 μL of each barcoded oligos (100 μM) were distributed into two 96-well plates. Forty-four microliters of Linker-R02 or Linker-R03 (12.5 μM, see Table 1) were then added to each well of the two plates. The plates were sealed and annealed in a thermocycler with the following program: 95° C. for 5 min, slowly cool down to 20° C. with a ramp of −0.1° C./s (stock plates). The stock solution plates were then divided into new 96-well plates, with each well of the working plates contains 10 μL of barcoded oligos ready for ligation reaction.

To prepare the barcoded RT primers (RNA barcode R01) 12.5 μL RNA_RE (# 01 to # 12, see Table 3) was pipetted into 12 tubes (final 100 μM) and mixed with 12.5 μL RNA_NRE (# 01 to # 12, matched with RNA RE, see Table 3, final 100 μM), and 75 pi H2O, and stored at −20° C.

To prepare P5 Adaptor mix for second adaptor tagging of DNA libraries, P5-FokI was mixed with P5c-NNDC-FokI, and P5H-FokI was mixed with P5Hc-NNDC-FokI (final concentration 50 μM for both, see Table 1). The oligo mixtures were then annealed in a thermocycler with the following program: 95° C. for 5 min, slowly cool down to 20° C. with a ramp of −0.1° C./s. The annealed P5 complex and P5H complex were then mixed on the ice at the ratio of 1:3, and stored at −20° C.

TABLE 1
Paired-Tag Primer Sequences. ddC = dideoxy cytosine modification;
* = phosphorothioate bond modification.

| SEQ ID | | |
|---|---|---|
| NO | Oligo name | Sequence (5′-3′) |
| 2 | pMENTs | 5Phos/CTGTCTCTTATACACATCTddC |
| 3 | AdaptorA | TCGTCGGCAGCGTCAGATGTGTATAAGAGACAG |
| 4 | Linker-R02 | CGAATGCTCTGGCCTCTCAAGCACGTGGAT |
| 5 | Blocker-R02 | ATCCACGTGCTTGAGAGGCCAGAGCATTCG |
| 6 | Linker-R03 | GGTCTGAGTTCGCACCGAAACATCGGCCAC |
| 7 | Quencher-R03 | GTGGCCGATGTTTCGGTGCGAACTCAGACC |
| 8 | Anchor-FokI- | AAGCAGTGGTATCAACGCAGAGTGAAGGATGTGGGGG |
| | GH | GGGG*H |
| | | (FokI recognition site underlined) |
| 9 | P5-FokI | ACACTCTTTCCCTACACGACGCTCTTCCGATCT |
| 10 | P5c-NNDC- | 5Phos/NNDCAGATCGGAAGAGCGTCGTGTAGGGAAAGA |
| | FokI | GTG |
| 11 | P5H-FokI | ACACTCTTTCCCTACACGACGCTCTTCCGATCTH |
| 12 | P5Hc-NNDC- | 5Phos/NNDCDAGATCGGAAGAGCGTCGTGTAGGGAAAG |
| | FokI | AGTG |
| 13 | PA-F | CAGACGTGTGCTCTTCCGATCT |
| 14 | PA-R | AAGCAGTGGTATCAACGCAGAGT |
| 15 | N5XX | AATGATACGGCGACCACCGAGATCTACACNNNNNNNN |
| | | TCGTCGGCAGCGTC |
| 16 | P7XX | CAAGCAGAAGACGGCATACGAGATNNNNNNGTGACTG |
| | | GAGTTCAGACGTGTGCTCTTCCGATC |
| 17 | P5 Universal | AATGATACGGCGACCACCGAGATCTACACTCTTTCCCT |
| | | ACACGACGCTCTTCCGATC*T |

Assembly of transposon complex

To prepare barcoded transposomes, barcoded DNA adaptor oligos (DNA barcode R01, DNA # 01 RE to DNA # 12 RE, see Table 2) were mixed with a pMENTs oligo (see Table 1) in twelve tubes, final concentration 50 μ.M. The oligo mixtures were then annealed in a thermocycler with the following program: 95° C. for 5 min, slowly cool down to 20° C. with a ramp of −0.1° C./s. One microliter of annealed transposome was then mixed with 6 μL of unloaded proteinA-Tn5 (0.5 mg/mL), briefly vortex and quickly spun down. The mixtures were incubated at room temperature for 30 min then at 4° C. for an additional 10 min. The transposon complex can be stored at 31 20° C. for up to 6 months.

To prepare the Tn5-AdaptorA, 25 pi Adaptor A (100 μM) were mixed with 25 μL pMENTs (100 μM). The mixture was heated for 5 min at 95° C. and slowly cooled down to 20° C. at the speed of 0.1° C./s. 1 μL of annealed transposome DNA was mixed with 6 μL of unloaded Tn5 (0.5 mg/mL), briefly vortexed and quickly spun down. The mixtures were incubated at room temperature for 30 min then at 4° C. for an additional 10 min. The mixtures were diluted 10 × with dilution buffer (10 mM Tris-HCl pH 7.5, 100 mM NaCl, 50% Glycol, 1 mM DTT), stored at −20° C.

TABLE 2
Barcoded DNA adaptor oligos. The recognition site for NotI (GCGGCCGC)
is underlined.

| SEQ ID | | |
|---|---|---|
| NO | Oligo Name | Sequence (5′-3′) |
| 18 | DNA_#01_RE | /5Phos/AGGCCAGAGCATTCGACATCGCGGCCGCAGA |
| | | TGTGTATAAGAGACAG |
| 19 | DNA_#02_RE | /5Phos/AGGCCAGAGCATTCGAATGAGCGGCCGCAGA |
| | | TGTGTATAAGAGACAG |
| 20 | DNA_#03_RE | /5Phos/AGGCCAGAGCATTCGAAGCTGCGGCCGCAGA |
| | | TGTGTATAAGAGACAG |
| 21 | DNA_#04_RE | /5Phos/AGGCCAGAGCATTCGAACAGGCGGCCGCAGA |
| | | TGTGTATAAGAGACAG |
| 22 | DNA_#05_RE | /5Phos/AGGCCAGAGCATTCGAGAATGCGGCCGCAGA |
| | | TGTGTATAAGAGACAG |
| 23 | DNA_#06_RE | /5Phos/AGGCCAGAGCATTCGATACGGCGGCCGCAGA |
| | | TGTGTATAAGAGACAG |
| 24 | DNA_#07_RE | /5Phos/AGGCCAGAGCATTCGATTACGCGGCCGCAGAT |
| | | GTGTATAAGAGACAG |
| 25 | DNA_#08_RE | /5Phos/AGGCCAGAGCATTCGAGTTGGCGGCCGCAGA |
| | | TGTGTATAAGAGACAG |
| 26 | DNA_#09_RE | /5Phos/AGGCCAGAGCATTCGACCGTGCGGCCGCAGA |
| | | TGTGTATAAGAGACAG |
| 27 | DNA_#10_RE | /5Phos/AGGCCAGAGCATTCGACGAAGCGGCCGCAGA |
| | | TGTGTATAAGAGACAG |
| 28 | DNA_#11_RE | /5Phos/AGGCCAGAGCATTCGATCTAGCGGCCGCAGAT |
| | | GTGTATAAGAGACAG |
| 29 | DNA_#12_RE | /5Phos/AGGCCAGAGCATTCGAGGGCGCGGCCGCAGA |
| | | TGTGTATAAGAGACAG |

Antibody staining and targeted tagmentation

To incubate the nuclei with antibodies, 3.6 million permeabilized nuclei were aliquoted into 12 Maximum Recovery tubes (300 k nuclei each), spun down at 1,000 g for 10 min and resuspended in 50 μL Complete Buffer (20 mM HEPES pH 7.5, 150 mM NaCl, 0.5 mM Spermidine, 1X Protease Inhibitor Cocktail 0.5 U/uL SUPERase IN (Rnase inhibitor), 0.5 U/uL RNase OUT (ribonuclease inhibitor), 0.01% IGEPAL-CA-630, 0.01% Digitonin and 2 mM EDTA). Antibodies (2 ug for each tube) were added and the mixture were rotated at 4° C. overnight. Antibodies: H3K4me1, H3K27ac, H3K27me3, H3K9me3. To wash out the unbound antibodies, the nuclei were spun-down at 600 g, 4° C. for 10 min, resuspended in 50 uL Complete Buffer, and repeated 1-2 times. The nuclei were again spun-down at 600 g, 4° C. for 10 min and resuspended in 50 μL Medium Buffer # 1 (20 mM HEPES pH 7.5, 300 mM NaCl, 0.5 mM Spermidine, 1 X Protease Inhibitor cocktail, 0.5 U/uL SUPERase IN, 0.5 U/uL RNase OUT, 0.01% IGEPAL CA-630, 0.01% Digitonin and 2 mM EDTA). Barcoded proteinA-Tn5 (# 01-# 12, 1 μL 0.5 mg/mL for each tube) were then added and the mixtures were rotated for 60 min at room temperature. Each tube received a proteinA-Tn5 loaded with a different barcode (comprising a restriction site for NotI, barcode round # 1, see Table 2). The nuclei were then spun down at 300 g, 4° C. for 10 min, and resuspended in 50 μL Medium Buffer # 2 (20 mM HEPES pH 7.5, 300 mM NaCl, 0.5 mM Spermidine, 1 × Protease Inhibitor cocktail, 0.5 U/uL SUPERase IN, 0.5 U/uL RNase OUT, 0.01% IGEPAL CA-630 and 0.01% Digitonin) and repeated for two additional times.

The tagmentation reaction was initiated by adding 2 μL 250 mM MgCl2 and was carried out at 550 r.p.m., 37° C. for 60 min in a ThermoMixer. The reaction was quenched by adding of 16.5 u.L 40.5 mM EDTA. Nuclei were then spun-down at 1,000 g, 4° C. for 10 min and proceeded to Reverse Transcription immediately.

Reverse Transcription

Nuclei pellets were resuspended in 20 μL RT Buffer in 12 tubes (1× Buffer RT, 0.5 mM dNTP, 0.5 U/μL SUPERase IN, 0.5 U/u.L RNase OUT, 2.5 μM barcoded T15 primer and 2.5 μM barcoded N6 primer (comprising a restriction site for Sbfl, barcode round # 1, see Table 3), and 1 U/μL Maxima Reverse H minus Reverse Transcriptase). The reverse transcription was performed in a thermocycler with the following program (Step 1: 50° C. x 10 min; Step 2: 8° C.×12 s, 15° C.×45 s, 20° C.×45 s, 30° C.×30 s, 42° C.×2 min, 50° C.×5 min, go to Step 2 for additional 2 times; Step 3: 50° C.×10 min and hold at 12° C.). After the reaction, the nuclei were transferred and pooled into a 1.5 mL Maximum Recovery tubes (on ice), pre-washed with 5% BSA in PBS and cooled on ice for 2 min, 4.8 μL of 5% Triton-X100. Nuclei were then spun-down at 1,000 g, 4° C. for 10 min and proceeded to ligation-based combinatorial barcoding immediately.

TABLE 3
Barcoded T15 primers and barcoded N6 primers. The recognition site for
SbfI (CCTGCAGG) is underlined.

| SEQ ID | | |
|---|---|---|
| NO | Oligo Name | Sequence (5′-3′)Sequence |
| 30 | RNA_#01_RE | /5Phos/AGGCCAGAGCATTCGTCATCCCTGCAGGTTTTT |
| | | TTTTTTTTTTTVN |
| 31 | RNA_#02_RE | /5Phos/AGGCCAGAGCATTCGTATGACCTGCAGGTTTTT |
| | | TTTTTTTTTTTVN |
| 32 | RNA_#03_RE | /5Phos/AGGCCAGAGCATTCGTAGCTCCTGCAGGTTTTT |
| | | TTTTTTTTTTTVN |
| 33 | RNA_#04_RE | /5Phos/AGGCCAGAGCATTCGTACAGCCTGCAGGTTTTT |
| | | TTTTTTTTTTTVN |
| 34 | RNA_#05_RE | /5Phos/AGGCCAGAGCATTCGTGAATCCTGCAGGTTTTT |
| | | TTTTTTTTTTTVN |
| 35 | RNA_#06_RE | /5Phos/AGGCCAGAGCATTCGTTACGCCTGCAGGTTTTT |
| | | TTTTTTTTTTTVN |
| 36 | RNA_#07_RE | /5Phos/AGGCCAGAGCATTCGTTTACCCTGCAGGTTTTT |
| | | TTTTTTTTTTTVN |
| 37 | RNA_#08_RE | /5Phos/AGGCCAGAGCATTCGTGTTGCCTGCAGGTTTTT |
| | | TTTTTTTTTTTVN |
| 38 | RNA_#09_RE | /5Phos/AGGCCAGAGCATTCGTCCGTCCTGCAGGTTTTT |
| | | TTTTTTTTTTTVN |
| 39 | RNA_#10_RE | /5Phos/AGGCCAGAGCATTCGTCGAACCTGCAGGTTTTT |
| | | TTTTTTTTTTTVN |
| 40 | RNA_#11_RE | /5Phos/AGGCCAGAGCATTCGTTCTACCTGCAGGTTTTT |
| | | TTTTTTTTTTTVN |
| 41 | RNA_#12_RE | /5Phos/AGGCCAGAGCATTCGTGGGCCCTGCAGGTTTTT |
| | | TTTTTTTTTTTVN |
| 42 | RNA_#01_NRE | /5Phos/AGGCCAGAGCATTCGTCATCCCTGCAGGNNNN |
| | | NN |
| 43 | RNA_#02_NRE | /5Phos/AGGCCAGAGCATTCGTATGACCTGCAGGNNNN |
| | | NN |
| 44 | RNA_#03_NRE | /5Phos/AGGCCAGAGCATTCGTAGCTCCTGCAGGNNNN |
| | | NN |
| 45 | RNA_#04_NRE | /5Phos/AGGCCAGAGCATTCGTACAGCCTGCAGGNNNN |
| | | NN |
| 46 | RNA_#05_NRE | /5Phos/AGGCCAGAGCATTCGTGAATCCTGCAGGNNNN |
| | | NN |
| 47 | RNA_#06_NRE | /5Phos/AGGCCAGAGCATTCGTTACGCCTGCAGGNNNN |
| | | NN |
| 48 | RNA_#07_NRE | /5Phos/AGGCCAGAGCATTCGTTTACCCTGCAGGNNNN |
| | | NN |
| 49 | RNA_#08_NRE | /5Phos/AGGCCAGAGCATTCGTGTTGCCTGCAGGNNNN |
| | | NN |
| 50 | RNA_#09_NRE | /5Phos/AGGCCAGAGCATTCGTCCGTCCTGCAGGNNNN |
| | | NN |
| 51 | RNA_#10_NRE | /5Phos/AGGCCAGAGCATTCGTCGAACCTGCAGGNNNN |
| | | NN |
| 52 | RNA_#11_NRE | /5Phos/AGGCCAGAGCATTCGTTCTACCTGCAGGNNNN |
| | | NN |
| 53 | RNA_#12_NRE | /5Phos/AGGCCAGAGCATTCGTGGGCCCTGCAGGNNNN |
| | | NN |

Ligation-Based Combinatorial Barcoding

Nuclei were resuspended and mixed in 1 mL 1× NEBuffer 3.1 and then transferred to Ligation Mix (2,262 μL H₂O, 500 μL 10× T4 DNA Ligase Buffer, 50 μL 10 mg/mL BSA, 100 μL 10× NEBuffer 3.1 and 100 μL T4 DNA Ligase). Each 40 μL of the ligation reaction mix was then distributed to Barcode-plate-R02 using a multichannel pipette and incubate at 300 r.p.m., 37° C. for 30 min in a ThermoMixer. 10 μL of R02-Blocking-Solution (264 μL of 100 μM Blocker-R02 oligo (see Table 1), 250 μL of 10× T4 Ligation Buffer, 486 μL ultrapure H₂O) was then added to each well using a multichannel pipette and the reaction were continued for an additional 30 min.

The nuclei were then pooled and spun-down at 1,000 g, 4° C. or 10° C. for 10 min.

The second round of ligation was then carried out similar to the first round in the barcode plate R03, except for after 30 min of the ligation reaction, Termination-Solution (264 μL of 100 μM R04 Terminator oligo (see Table 1), 250 μL A of 0.5 M EDTA and 236 μL ultrapure H₂O) was added to quench the reaction.

All nuclei were combined in a 15 mL tube (pre-washed with 0.5% BSA) and spun-down at 1,000 g, 10° C. for 10 min. The supernatant was discarded. The nuclei were washed once with cold PBS and spun-down at 1,000 g, 10° C. for 10 min and resuspended in 200 μL-1 mL cold PBS (optimal concentration 1,000 cell/μL). The samples were ready for lysis and DNA Cleanup.

Nuclei lysis

Typically, 100,000 to 300,000 nuclei could be recovered after ligation-based barcoding. Nuclei were then resuspended in PBS, counted and aliquot to sub-libraries containing 2 k to 5 k nuclei or 2 k to 4 k nuclei (optimal ˜2.5 k nuclei per tube). Aliquoted nuclei could be stored at -80° C. for up to 6 months.

Sub-libraries were diluted to 35 μL with PBS. 5 μL 4M NaCl, 5 μL 10% SDS and 5 μL 10 mg/mL Protease K was then added and nuclei were lysed at 850 r.p.m., 55° C. for 2 h or overnight in a ThermoMixer. The lysed solution was cooled to room temperature and then purified with 1× paramagnetic SPRI beads and eluted in 12.5 μL H₂O. As much SDS as possible was removed. The purified DNA can be stored at −20° C. or −80° C. for up to 6 months.

TdT-Tailing and Pre-Amplification of Barcoded DNA/cDNA

Polynucleotide tailing of cDNA with terminal deoxynucleotidyltransferase (TdT) results in the addition of a homopolymeric sequence at its 3′-end that can then be used as an anchor for amplification. 1.5 μL 10X TdT buffer, 0.5 μL 1 mM dCTP was added into 12.5 μL purified DNA/cDNA mix and denatured at 95° C. for 5 min and then quickly chilled on ice for 5 min. 1 μL of TdT was added and incubated at 37° C. for 30 min followed by heat deactivation at 75° C. for 20 min. Anchor Mix (6 μL 5× KAPA Buffer, 0.6 μL 10 mM dNTPs, 0.6 μL 10 μM Anchor-FokI-GSH-Oligo (see Table 1) and 0.6 μL KAPA high fidelity hot start polymerase were added and the linear amplification was performed in a thermocycler with the following program (Step 1: 95 or 98° C.×3 min; Step 2: 95 or 98° C.×15 s, 47° C.×60 s, 68° C.×2 min, 47° C.×60 s, 68° C.×2 min and repeat Step 2 for additional 15 times; Step 3: 72° C.×10 min and hold at 12° C.).

Preamplification Mix (4 μL 5X KAPA buffer, 0.5 μL 10 mM dNTPs, 2 μL of 10 uM of primers PA-F and PA-R (see Table 1), 0.5 μL KAPA high fidelity hot start polymerase were then added and pre-amplification was performed in a thermocycler with the following program (Step 1: 98° C.×3 min; Step 2: 98° C.×20 s, 65° C.×20 s, 72° C.×2.5 min and repeat Step 2 for additional 9-10 times; Step 3: 72° C.×2 min and hold at 12° C.). Amplified products were purified with paramagnetic SPRI bead double-size selection (10 μL+37.5 μL, 0.2 X +0.75X) and were eluted in 35 pi H₂O. Typical concentrations were 1-30 ng/μl. Purified DNA could be stored at −20° C. or −80° C. for up to 6 months.

Endonuclease digestion and second adaptor tagging

During tagmentation and RT, a Sbfl restriction site was introduced into the RNA library and a NotI restriction site was introduced into the DNA library. The DNA library was generated by digesting the RNA library with Sbfl. The RNA library was generated by digesting the DNA library with NotI.

17 pi each of purified amplified products were transferred into two tubes for DNA and RNA library construction, respectively. Add 2.5 μL 10X Cutsmart buffer, 1 μL Sbfl-HF and 1 μL FokI and 3.5 μL H₂O to DNA-tube. Add 2 μL 10X Cutsmart buffer and 1 μL NotI-HF to RNA-tube. The digestion reaction was incubated at 37° C. for 60 min. Use 1.25 X (31.3 μL for DNA and 25 μL for RNA) SPRI beads to purify the digestion product and elute in 10 μL. Purified DNA could be stored at −20° C. or −80° C. for up to 6 months.

For the DNA part, 2 μL 10X T4 DNA Ligase Buffer, 2 μL P5 Adaptor Mix, 4 μL H₂O and 2 μL T4 DNA Ligase were added and ligation reaction were carried out in a thermocycler with the program (4° C. for 10 min, 10° C. for 15 min, 16° C. for 15 min, 25° C. for 45 min). The ligation product was then purified with 1.25X (25 μL) SPRI beads and elute in 30 μL H₂O. Purified DNA could be stored at −20° C. or −80° C. for up to 6 months

For the RNA part, add 10.5 μL 2X TB and 0.5 μL 0.05 mg/mL Tn5-AdaptorA were added and tagmentation reaction were carried out at 550 r.p.m., 37° C. for 30 min in a ThermoMixer followed by cleaned up using QlAquick PCR purification kit and eluted in 30 μL 0.1X elution buffer.

Indexing PCR and Sequencing

The PCR mix was prepared by mixing 30 μL purified P5-tagged product, 10 μL 5X Q5 buffer, 1 μL 10 mM dNTP, 0.5 _{I}A 50 μM P5 Universal primer for DNA or N5 primer for RNA, 2.5 μL 10 μM P7 primer (see Table 1), 5 μL H₂O and 1 μL NEB Q5 DNA Polymerase.

The PCR program for DNA libraries used was: Step 1: 98° C.×3 min; Step 2: 98° C.×10 s, 63° C.×30 s, 72° C.×1 min; repeat Step 2 for 8 cycles; Step 3: 72° C.×1 min; Step 4: hold at 12° C.

The PCR program for RNA libraries used was: Step 1: 72° C.×5 min, 98° C.×30 s; Step 2: 98° C.×10 s, 63° C.×30 s, 72° C.×1 min and repeat Step 2 for additional 8-13 times to reach 10 nM concentration; Step 3: 72° C.×1 min; Step 4:hold at 12° C.

Library cleanup was performed using 0.9 X (454) SPRI beads. Purified libraries could be stored at -20° C. or -80° C. for up to 6 months.

Sequencing

The final libraries were multiplexed and sequenced with standard Illumina sequencing primers on commercial sequencing platforms, including, for examplea NextSeq 550, NextSeq 1000/2000,NovaSeq 6000, or HiSeq 2500/4000 platforms. Libraries were loaded at recommended concentrations according to manufacturer's instructions. At least 50 and 100 sequencing cycles are recommended for Readl and Read2, respectively. For example: using PE 50 (or 53) +7 +100 cycles (Readl +Index 1 +Read2) on a NextSeq 500 platform with 150-cycle sequencing kits, or PE 100 +7 +100 cycles on a NovaSeq 6000 platform with 200-cycle sequencing kits.

Data Analysis Procedures

Pre-Processing of Paired-Tag Data

Initial Paired-Tag data processing included (a) extracting barcode sequences from Read2, (b) assigning barcodes combinations to cellular barcodes references (assign barcode sequences to ID of 12 sample tubes and 2 rounds of 96 wells), (c) mapping the assigned reads to reference genome and (d) generating cell-to-features matrices for downstream analyses.

The following metrics during initial Paired-Tag data processing can be used for quality control. For step 2(a), typically >85% and >75% of DNA and RNA reads will have full ligated barcodes. For step 2(b), >85% of both DNA and RNA reads can uniquely assigned to one cellular barcode with no more than 1 mismatch. For step 2(c), typically >85% of assigned reads can be mapped to the reference genome; depending on which histone mark targeted, from 60% to >95% of assigned DNA reads can be mapped to the reference genome.

Cellular barcodes and the linker sequences were read by Read2. The first base of BC# 1, BC# 2 and BC# 3 should locate within 84-87^{th}, 47-50^{th} and 10-13r^{d} base of Read2. The positions of barcodes were identified by matching the linker sequences adjacent to the cellular barcodes. Readl and Read2 of each library were paired to generate a single new FASTQ file by joining read sequence (read sequence of Readl and UMI [first 10 bps of Read2 sequence]) and quality values into Linel and joining the 3 rounds of barcodes sequences as well as the quality values into Line 2 and Line 4. A bowtie reference index was generated with all possible cellular barcode combinations (96*96*12). The combined FASTQ files contains barcodes sequences were then mapped to the cellular barcodes reference using bowtie (Langmead & Salzberg, Nat Methods 9, 357-359) with parameters: -v 1 -m 1 --norc (reads with more than 1 barcode mismatch and can be assigned to more than 1 cell were discarded). The resulting SAM file was then converted to a final FASTQ file by using adding RNAME (of SAM file) into Linel and extract the original Readl sequence and quality values from QNAME (of SAM file) into Line2 and Line4 of the final FASTQ file. NextEra adaptor sequences were trimmed from 3′ of DNA and RNA libraries, Poly-dT sequences were further trimmed from 3′ of RNA libraries and low-quality reads (L=30, Q=30) were excluded for further analysis.

Analysis of Paired-Tag Data

Evaluation of collision rate: Reads from species mixing test were extracted based on cellular barcodes (BC# 1=06 or 12) and mapped to a reference genome using STAR version: 2.6.0a (Dobin & Gingeras, Curr Protoc Bioinformatics 51, 11 14 11-19) with the combined reference genome (GRCh37 for human and GRCm38 for mouse). Duplicates were removed based on the mapped position, cellular barcode, PCR index and UMI. For evaluation of the collision rate, nuclei with less than 80% UMIs mapped to one species were classified as mixed cells.

Reads mapping: Cleaned reads were first mapped to a mouse GRCm38 genome reference genome with STAR (version: 2.6.0a) for RNA or bowtie2 for DNA. Mapped DNA reads of H3K4me1, H3K27ac and H3K27me3 were further filtered by mapping quality (MAPK>10). Duplicates were removed based on the mapped position, cellular barcode, PCR index and UMI. BC# 1 was used for the identification for the origin of samples. Low coverage nuclei were removed from further analysis (<1,000 transcripts and <500 unique DNA reads). Before generating the cell-counts matrices, DNA bam files were further filtered by removing high-pileup positions (cutoff=10) regardless of cellular barcode, PCR index and UMI.

Clustering of Paired-Tag profiles: RNA alignment files were converted to a matrix with cells as columns and genes as rows. DNA alignment files were converted to a matrix with cells as columns and 5-kb bins (instead of peaks) as rows. Cells with less than 200 features in both DNA and RNA matrices were removed. DNA matrix was further filtered by removing the 5% highest covered bins. Clustering of single-cells based on RNA-profiles was performed with Seurat package (Stuart et al. Cell 177, 1888-1902, e1821 (2019). Briefly, cell-to-gene counts were normalized and variable genes were selected for dimension reduction by PCA, batch effects were corrected with harmony (Korsunsky et al. Nat Methods 16, 1289-1296), visualized with UMAP and clustered with Louvain algorithm. Cell groups with high expression levels of marker genes from multiple major cell types were considered as doublets and excluded from further analyses. Co-embedding of Paired-Tag RNA profile and published scRNA-seq dataset (Zeisel et al. Cell 174, 999-1014, e1022) were performed using Seurat package. To compare the clustering results from different studies, overlap coefficients (0) were calculated according to the number of cells with label from Paired-Tag dataset (A), from Zeisel Cell, 2018⁵³ (B) and from co-embedding (C):

$${O}_{i , j} = min ( max ( \frac{{A}_{i} ⋂ {C}_{x}}{{A}_{i}} ) , max ( \frac{{B}_{j} ⋂ {C}_{x}}{{B}_{j}} ) )$$

To visualize the single-cell DNA profiles, cell-to-bins (5-kbp bin-size) matrices were converted to cell-to-cell similarity Jaccard matrices by snapATAC (Fang et al. bioRxiv, 615179 (2019)), followed by dimension reduction by PCA, batch effect correction with harmony and visualization with UMAP. To compare the clustering results from RNA and DNA based analysis, Jaccard overlap coefficients (J) were calculated according to the number of cells with label from RNA clustering (R) and DNA clustering (D):

$${J}_{i , j} = \frac{{D}_{i} ⋂ {R}_{j}}{{D}_{i} ⋃ {R}_{j}}$$

Classification of Promoter and CRE Modules

To classify genes according to epigenetic states of promoters, gene expression (RPKM) and reads densities of promoters (CPM) were summarized from aggregated profiles based on transcriptome-based clustering. Genes with RPKM >1 for expression and CPM>1 for promoters in at least one cluster were retained for analysis. Genes were first grouped by K-means clustering based on reads densities of 4 histone marks (k=4). Each group was then subjected to secondary K-means clustering based on gene expression, resulting in 7 promoter groups.

To classify CRE into different groups, first, the cCRE list was from CEMBA (Li, et al, bioRxiv, 2020.2005.2010.087585 (2020)) and extended for 1,000 bp (500 bp at both directions). cCRE overlap with promoter regions (−1,500 bp to +500 bp of TSS) were excluded for further analysis. CRE reads densities of four histone marks were then summarized from aggregated profiles based on transcriptome-based clustering. cCREs with CPM>1 in at least one cluster or one histone profile were retained for analysis. Promoters were first grouped by K-means clustering based on reads densities of 4 histone marks (k=4). Each group was then subjected to secondary K-means clustering based on H3K27ac reads densities, resulting in 8 CRE groups.

Motif Enrichment and Gene Ontology Analysis

Motif enrichment for each cell type: Motif enrichment for each cell type and histone modifications were carried out using ChromVAR (Schep et al., Nat Methods 14, 975-978 (2017).). Briefly, mapped reads were converted to cell-to-bin matrices with a bin-size of 1,000 bp for four histone profiles. Reads for each bin were summarized from all cells of the same groups from transcriptome-based clustering. GC bias and background peaks were calculated and motif enrichment score for each cell type was then computed using the computeDeviations function of ChromVAR.

Motif enrichment for each CRE module: Motif enrichment for each CRE module was analyzed using Homer (v4.11, Heinz et al. Mol Cell 38, 576-589 (2010)). A region of +/−200 bp around the center of the element was scanned for both de novo and known motif enrichment analysis. The total peak list was used as the background for motif enrichment analysis of cCREs in each group.

Gene ontology enrichment: Gene ontology annotation was performed with Homer (v4.11) with default parameters. Gene set library “Biological process” was used. GO terms with more than 500 total genes in the list were excluded from the “Top Enriched GO Terms”.

Linking CREs with putative target genes

To predict putative target genes for active and repressive cCREs, first the candidate CRE-gene pairs were identified by calculating the co-occupancy of H3K4me1 reads between promoter regions (-1,500 bp to +500 bp) and cCREs with cicero (Pliner et al. Mol Cell 71, 858-871, e858, (2018).) using default parameters. cCRE-gene pairs with co-accessibility of >0.1 were used for further analysis.

To identify functional cCRE-gene pairs, the Spearman's correlation coefficients were then calculated between H3K27ac (for active pairs) or H3K27me3 (for repressive pairs) reads densities of cCREs (CPM) and gene expression of corresponding linked genes (RPKM) across clusters from transcriptome-based clustering. To estimate the background noise levels, the cell IDs were shuffled for each read and calculated the corresponding Spearman's correlation coefficients. False-positive detection rates were estimated based on the fraction of detected pairs from the shuffled group under different cutoffs. Finally, a cutoff of FDR<0.05 was used for the identification of both active and repressive cCRE-gene pairs.

External Datasets

CEMBA dataset were available from NEMO (https://nemoanalytics.org) with accession number of RRID SCR 016152.

ENCODE (https://www.encodeproject.org/) datasets were downloaded with the accession numbers: H3K4mel (ENCSROOOAPW), H3K27ac (ENCSR000A0C), H3K27me3 (ENCSR000DTY), H3K9me3 (ENCSR000AQ0), DNase-seq (ENCSR959ZXU).

The other external datasets were downloaded from NCBI Gene Expression Omnibus (GEO) (http://www.ncbi.nlm.nih.gov/geo/), with the accession numbers: SPLiT-seq (GSE110823), CoBATCH (GSE129335), itChIP (GSE109762) and HT-scChIP-seq (GSE117309).

10× scRNA-seq datasets were download from 10× genomics website (https://www.10xgenomics.com/).

Results

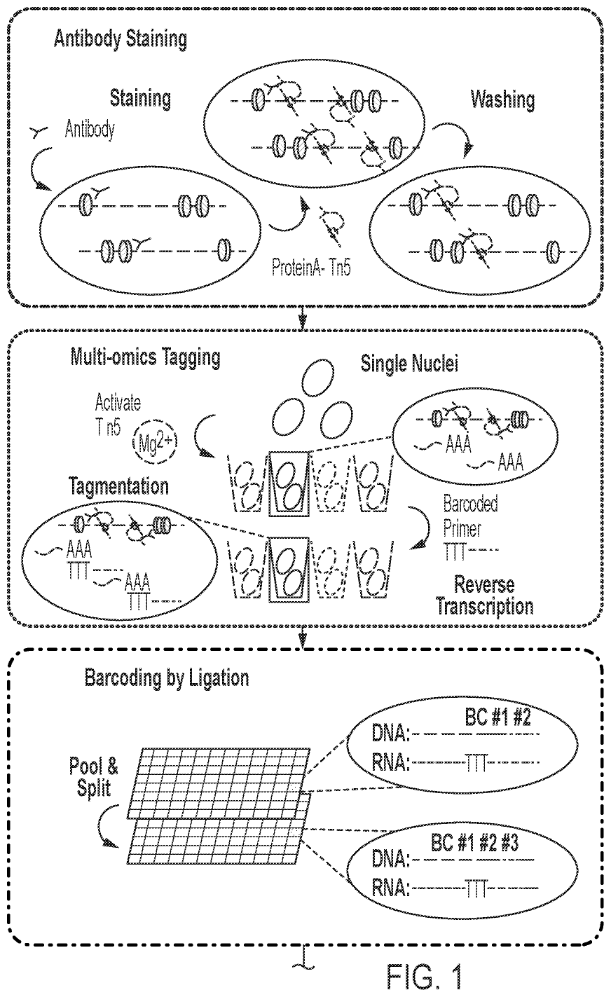

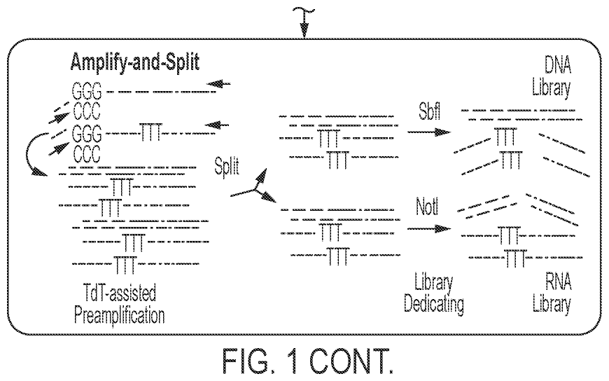

Disclosed herein is a method called Paired-Tag (parallel analysis of individual cells for RNA expression and DNA from targeted tagmentation by sequencing). First, permeabilized nuclei were incubated with antibodies targeting specific histone modifications. Afterwards, the nuclei were incubated with protein A-fused Tn5, which was loaded with an adaptor including a barcode and a NotI restriction site. Protein A allowed the targeting of Tn5 to the chromatin sites of interest (FIG. 1). The reactions were carried out in 12 different wells, each with a well-specific DNA barcode included in the transposase adaptors and RT primers, to label different samples or replicates (first round of barcodes). Tagmentation was initiated, resulting in DNA fragments comprising the first barcode and the NotI restriction site. Then, reverse transcription (RT) was performed using primers comprising the same barcode and a Sbfl restriction site, resulting in cDNA molecules comprising the same barcode as the DNA fragments located within the same cell as well as the Sbfl restriction site. At this point, the nucleic were still intact and comprised DNA and cDNA each tagged with one of twelve barcodes.

Next, a ligation-based combinatorial barcoding strategy was used to introduce the second and third rounds of DNA barcodes to the nuclei, by sequentially attaching well-specific DNA barcodes to the 5′-end of both chromatin DNA fragments and cDNA from RT in 96-well plates. First, the twelve samples from round 1 were pooled and added to a 96 well plate comprising 96 different barcodes (second round of barcodes). The samples were pooled and added to a second 96 well plate comprising 96 different barcodes (third round of barcodes). Finally, the barcoded nuclei were divided into sub-libraries and lysed, and the chromatin DNA and cDNA were purified.

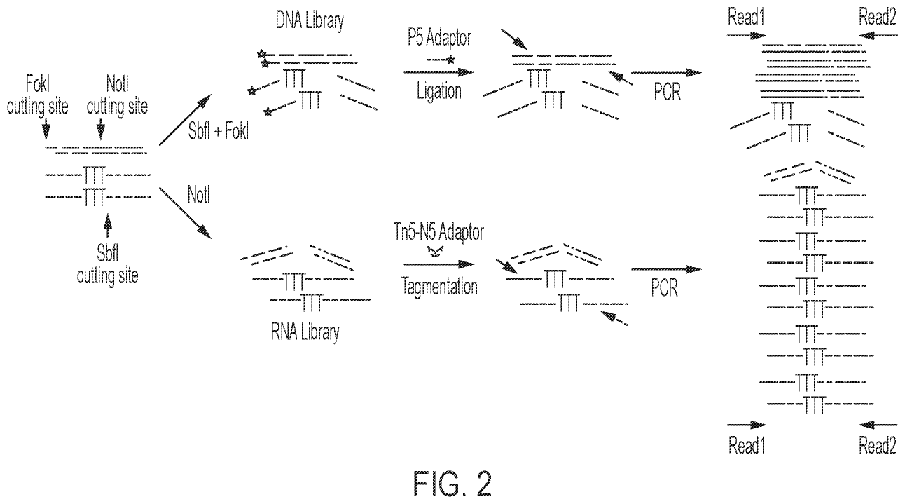

The DNA and the RNA library were prepared for sequencing using an “amplify-and-split” strategy (see FIGS. 1 and 2). The isolated DNA and cDNA were subjected to polynucleotide tailing with terminal deoxynucleotidyltransferase (TdT), resulting in the addition of a homopolymeric sequence at its 3′-end that was then used as a template for amplification. The primer used for the amplification of the polynucleotide tailed DNA comprised a restriction site for FokI.

To obtain the RNA library, the pool of DNA and cDNA was digested with NotI. Tn5 transposases bound to the second sequencing adaptor were used to add the second sequencing adaptor.

The fragment sizes of DNA from targeted tagmentation were shorter than those of cDNA from RT, which would result in lower library yields if Tn5 tagmentation was used to add the second adaptor. Therefore, to obtain the DNA library, the pool of DNA and cDNA was digested with FokI and Sbfl. FokI, a type IIS endonuclease, created a nick and the second sequencing adaptor was then introduced by ligation.

To benchmark the efficiency of Paired-Tag, 10,000 HeLa cells were contacted each with antibodies against H3K4me1, H3K27ac, H3K27me3 and H3K9me3. The aggregate profiles of each histone modification were compared with published ChIP-seq datasets of this cell line (Thurman et al. Nature 489, 75-82 (2012).). The enriched regions from Paired-Tag experiments overlapped quite well (65.9% for H3K4me1, 65.7% for H3K27ac, 59.6% for H3K27me3 and 64.0% for H3K9me3) with those from the published ChIP-seq datasets for all four histone marks. The genome-wide distribution of each histone mark also correlated well with the published datasets (Pearson's correlation coefficients 0.70-0.86 for different histone marks). The gene expression levels measured from Paired-Tag were highly correlated with in-house generated nuclei RNA-seq from the same cell line (Pearson's correlation coefficient 0.96). These data confirm that the Paired-Tag can provide comparable chromatin and transcriptome information with ChIP-seq and RNA-seq from bulk-cell samples.

Single-cell co-assay of histone marks and transcriptome in mouse cortex and hippocampus by Paired-Tag

To demonstrate the utility of Paired-Tag for analysis of heterogeneous tissues, the method was applied to freshly collected frontal cortex and hippocampus tissues from adult mice, focusing on the four aforementioned histone marks. The aggregated single-cell Paired-Tag DNA profiles and bulk profiles generated in parallel showed an excellent agreement (Pearson's correlation coefficients 0.72-0.96) for different histone marks. Paired-Tag generated datasets with high mapping rates: >95% of H3K4me1 and H3K27ac reads, ˜72% of H3K27me3 reads, and >85% of H3K9me3 and RNA reads can be mapped to the reference genome. To estimate the library complexities of Paired-Tag datasets, a fraction of representative nuclei was sequenced to near saturation (˜80% PCR duplication rates). It was found that Paired-Tag profiles resulting from random barcode collision was less than 5%, estimated from the human/mouse mixed samples. Up to 20,000 unique loci per nucleus were recovered for DNA profiles (medium numbers per nucleus, H3K4me1: 19,332 and 17,357, H3K27ac: 4,460 and 4,543, H3K27me3: 2,565 and 2,499, H3K9me3: 16,404 and 18,497, for frontal cortex and hippocampus, respectively) and up to 15,000 UMI per nucleus for RNA profiles (median numbers, 14,295 and 8,185 UMIs, corresponding to 2,400 and 1,855 genes, for frontal cortex and hippocampus, respectively. The “amplify-and-split” strategy of Paired-Tag reduced the risk of losing materials during the process of measuring multiple molecule types, and provided both DNA and RNA datasets at comparable library complexities as stand-alone high-throughput scChIP-seq and scRNA-seq assays.

Epigenome maps of cortical and hippocampal cell types in adult mice

Next, a total of 65,000 nuclei were sequenced to moderate depth (duplication rates: ˜40-60%). After filtering out nuclei with low sequence coverage or due to potential doublets (see Methods above), 45,446 nuclei were recovered with matching DNA and RNA Paired-Tag profiles, with 941-7,477 unique DNA loci mapped per nucleus for different histone marks or brain regions (medium numbers, H3K4me1: 6,073 and 5,799, H3K27ac: 1,942 and 1,949, H3K27me3: 941 and 942, H3K9me3: 6,765 and 7,477, for frontal cortex and hippocampus, respectively), as well as 5,698 and 4,039 RNA UMI per nucleus (median 1,290 and 992 genes per nucleus) for frontal and hippocampus, respectively. These nuclei were clustered into 22 cell groups based on their transcriptome profiles using the Seurat package. The variable genes were first selected for dimensional reduction with Principal Component Analysis (PCA), followed by Uniform Manifold Approximation and Projection (UMAP) and graph-based Louvain clustering. Based on marker genes expression, the 22 cell groups were assigned to seven cortical neuron types (Snap25+, Satb2+, Gadb1−), four hippocampal neuron types (Snap25+, Slc 1 7a7+or Proxl+), three inhibitory neuron types (Gadb1/Gad2+) and eight non-neuron cell types (Snap25−) including oligodendrocyte precursor cells (OPC), two groups of oligodendrocytes (OGC), two groups of astrocytes (ASC), microglia, endothelial and choroid plexus: with equivalent fractions from each biological replicate for all the clusters. The Paired-Tag transcriptomic profiles were also compared with previously published scRNA-seq datasets from the same brain regions (reference dataset, Zeisel et al. Cell 174, 999-1014, e1022 (2018).) and excellent agreement was found. Specifically, 16 of the 22 clusters can be uniquely assigned to a corresponding cluster (or several closely-related sub-clusters) from the reference datasets. Some of the sub-clusters here matched multiple sub-clusters of the reference dataset, which includes: the CA1 and subiculum clusters in our datasets fell into two CA1 neuron groups (TEGLU21, 23), 2 OGC cell clusters matched with oligodendrocytes groups (MFOL, MOL) and 2 ASC cell clusters aligned with the two astrocyte groups (ACNT1, 2) of the reference dataset.

The Paired-Tag profiles were also clustered based on DNA profiles of different histone marks using the SnapATAC package (Fang et al, bioRxiv, 615179 (2019)). Cell-to-bins DNA matrices were converted to cell-to-cell Jaccard similarity matrices followed by dimension reduction using PCA and graph-based clustering. For H3K4me1- and H3K27ac-based clustering, 18 and 16 clusters were revealed, respectively. 15 groups of H3K4me1-based and 14 of H3K27ac-based clustering matched well with those from RNA. Two cortical neuron clusters (L4 and L5) in H3K4me1- and H3K27ac-based clustering matched with L4, L5a and L5 groups of RNA-based clustering; and the Subiculum group in H3K4me1-based clustering fell into CAL Subiculum and CA2/3 groups of RNA-based clustering. For H3K27me3-based clustering, all cortical excitatory neurons formed a single cluster distinct from all the other cell groups. For H3K9me3, only the major non-neuron cell types can be separated, while all neuronal cell types were grouped together as a single cluster. These results indicate that cell-clustering based on Paired-seq profiles varies considerably depending on the histone marks used, and repressive histone marks do not resolve the cell types as well as the active histone marks.

The inconsistency of cell clustering based on different histone marks individually indicates that it is important to use the transcriptome profiles to construct the cell-type-specific epigenome maps. Genome-wide maps of each histone modification were generated long with gene expression profiles in each of the 22 mouse brain cell types identified based on transcriptome information of the Paired-Tag datasets.

Integrative analysis of chromatin state and gene expression at gene promoters across different brain cell types

To investigate the relationship between chromatin states and cell-type-specific gene expression, the Paired-Tag signals of each histone modification at the gene promoter regions (-1,500 bp to +500 bp) in the brain cell types were aggregated. For this analysis, the 18 cell groups with at least 50 cells and at least 50,000 combined unique reads for all the five modalities were mainly examined. A total of 17,398 genes (GENCODE GRCm38.p6) with sufficient levels of transcription (RPKM >1) or promoter occupancy (CPM >1 for histone marks in at least one cell group) were retained for subsequent analysis. Using K-means clustering, these gene promoters were categorized into seven groups with distinct combinations of histone modification: class I promoters appeared to be repressed by H3K9me3 (13.1% of all tested genes), class II-a and II-b groups were associated with the polycomb repressive histone mark H3K27me3 (9.2% of all tested genes), and the rest four groups were associated with variable levels of active histone marks H3K4me1 and H3K27ac (77.6% of all tested genes). Expression levels of class I and II genes were negatively correlated with the repressive histone marks H3K9Kme3 or H3K27me3, while expression levels of class III genes were positively correlated with the active histone marks H3K4me1 and H3K27ac at promoter regions.

Gene Ontology (GO) analysis was carried out and distinct functional categories of genes within each group were found. For example, genes in class I were strongly enriched for sensory-related pathways, including olfactory receptor (OR) genes (Olfr, 647 of 730 detected) and vomeronasal (Vmnr, 189 of 201 detected) receptor genes. OR genes were previously shown to be marked in a highly dynamic pattern with constitutive heterochromatin marks during the process of OR choice in olfactory sensory neurons. The data suggest OR genes were also silenced in frontal cortex and hippocampus by heterochromatin. H3K27me3-repressed genes can be further divided into two groups: class II-a genes were repressed in all cell clusters and class II-b genes repressed in a more restricted manner. GO analysis revealed that II-a group genes were enriched for terms involved in general developmental processes such as pattern specification process and embryonic organ development, while II-b group genes were enriched for terms including morphogenesis of an epithelium. Genes in II-b include those with function in differentiation of glial cells, such as Sox10 and NotchI. Genes in III-a group were characterized by active chromatin state at promoters in all cell types (10.4% of class III genes), while genes in III-b group were expressed in all neuronal cell types (5.9% of class III genes) and genes in III-c group were glial-expressed (31.0% of class III genes). Group III-d genes (52.6% of class III genes) were marked by active chromatin state in a cell-type-specific manner, with corresponding cell-type-specific expression patterns. These genes were enriched for GO terms with more specific cellular processes: for example, hippocampal neuron-expressed genes were enriched for learning or memory and microglia-expressed genes were enriched for inflammatory response. These results demonstrate the key role of H3K27me3 in defining major types during development processes and the contribution of H3K27ac to diverse expression patterns across sub-cell-types in the mouse brain.

Integrative Analysis of Chromatin State at Distal Elements Across Brain Cell Types

Cis-regulatory elements (CREs) are marked with highly cell-type-specific chromatin states and strongly correlated to cell-type-specific gene expression. Recently, a comprehensive analysis of chromatin accessibility from the adult mouse cerebrum identified 491,818 candidate CREs (cCREs) (Li et al. bioRxiv, 2020.2005.2010.087585 (2020). It was found that 286,168 (58.2%) distal CREs from this list showed sufficient levels of Paired-Tag signals in at least one cell group and one or more histone marks (CPM >1, and more than 1,500 bp upstream and 500 bp downstream away from transcription start sites, TSS). To characterize the chromatin state of these candidate CREs across different brain cell types, K-means clustering was performed with the aggregate Paired-Tag signals of different histone marks in each of the 18 cell clusters defined above. These candidate CREs as categorized into 8 groups: two were marked by H3K9me3 in either all cell clusters (class eI-a, 16.3% of all CREs) or selectively in neuronal cells (class eI-b, 4.9% of all CREs), two were marked with H3K27me3 (ell-a, 5.5% and eII-b, 3.1% of all CREs) primarily in all neuronal cell clusters or in a more restricted manner (eII-b elements). The rest four groups (class eIII-a to eIII-d) were marked by variable levels of H3K4me1 and H3K27ac modifications in different cell clusters. Similar to the promoter groups, the sub-class of cCREs with H3K27ac mark in one or a few cell groups comprised the largest fraction (class eIII-d, 37.1% of all CREs). cCREs with different histone modifications distribute differently in the genome. For example, H3K9me3-marked cCREs reside preferentially in intergenic regions (eI-a and eI-b), while cCREs marked by relatively invariable H3K4me1 and H3K27ac levels tend to reside in genic regions (eIII-a). Class eII-b cCREs were significantly enriched for CpG islands (CGI) regions (5.4%, p <2.2x10⁻¹⁶) and ell-a cCREs were less enriched (2.0%, p=0.002). The two H3K9me3-marked groups were depleted from CGI regions (0.16% and 0.12%, p <2.2×10⁻¹⁶). For the active cCRE groups, class eIII-a cCREs displayed the highest enrichment for CGI regions (14.1%, p <2.2 x10⁻¹⁶) while the other sub-classes of eIII cCREs were not.

To identify potential transcription factors that act on the above classes of cCRE, motif enrichment analysis was performed with the JASPAR database (Khan et al. Nucleic Acids Res 46, D260-D266 (2018). The heterochromatin eI-a group were enriched for motif of EVX1, a transcriptional repressor during embryogenesis; class eI-b cCREs were also enriched for the motif of a well-known repressor MAFG, which is expressed in central nervous system and dysregulation of this regulator can lead to neuronal degeneration phenotypes. The two polycomb-repressed cCRE groups were both enriched for LHX motifs, however, Genomic Regions Enrichment of Annotations Tool (GREAT) analysis revealed distinct GO terms for them: the eII-a group were strongly enriched for general cellular processes such as the term: transcription from RNA polymerase II promoter, while the class ell-b cCREs were enriched for developmental processes including the sensory organ development. The group eIII-d with dynamic H3K27ac across all clusters were enriched for CTCF motif, supporting the role of enhancer-promoter looping in regulating gene expression across multiple cell types. Enrichment analysis of known TF motifs followed by K-means clustering also revealed distinct modules. The ell-a group were enriched for motifs such as LHX, Nanog and Isll. The eIII-b pan-neuron group was enriched for neurogenic factors, such as MEF2 and NEUROD. The pan-glia group (eIII-c) was enriched for motifs recognized by FOX, SOX, and ETV family transcription factors, with the latter two also enriched in the oligodendrocyte- or microglia-specific groups in e111-d. The heterochromatin el-a group and inhibitory neuron groups in eIII-d were enriched for Ascll motif. Ascll can function as a pioneer factor targeting closed chromatin to activate the neurogenic gene expression programs as well as to induce the generation of GABAergic neurons.

The joint profiles of chromatin state and transcriptome across diverse brain cell types provide an excellent opportunity to infer potential regulators for each cell lineage. The TF motif enrichments in cCREs identified in each cell group were calculated using ChromVAR, and their correlation compared with expression levels of the corresponding TF genes. More than half of the TFs (65%) showed a positive correlation between gene expression levels and corresponding motif enrichment in the cCREs in the cell type, including 51 high-confident TFs that showed significant concordances (FDR <0.1) for both H3K4me1 and H3K27ac. For example, one of the top-ranked TFs, Fli 1 , was restricted in microglia and endothelial cells. Fli 1 is known to activate chemokines to mediate the inflammatory response in endothelial cells and recently found to be in a coordinated gene expression module associated with Alzheimer's disease. Other highly ranked TFs including Sox9/10, Mef2c and Neurod2, etc, known to play a critical role in the development of neuronal systems.

Integrative Analysis of Chromatin State and Gene Expression Connects Distal Candidate Cres to Putative Target Genes

Distal regulatory elements including enhancers and silencers control cell-type-specific transcriptional programs during development or in response to stimuli. Imaging-based tools and chromosome conformation capture techniques have been extensively used to elucidate the interplay between promoters and distal CREs. The epigenetic and transcriptional states from the same cells provide an excellent opportunity to connect both the active and repressive cCREs to their putative target genes. First putative promoter-CRE pairs were identified based on co-occupancy of H3K4me1 reads between cCRE and TSS-proximal regions (-1,500 bp to +500 bp) across all cells using Cicero. Then, the pairwise Spearman's correlation coefficients (SCC) were calculated between the gene expression levels of the putative target genes and the histone mark levels of the cCREs across cell clusters.

32,252 candidate CRE-gene pairs were identified where H3K27ac levels at the distal cCREs positively correlated with gene expression, and 15,199 pairs of candidate CRE-gene where H3K27me3 levels at the cCREs negatively correlated with expression of linked genes (FDR <0.05). The finding of both active and repressive cCREs provide additional insight into the mechanism of gene regulation in these brain cell types. A significant fraction of positive cCRE-gene pairs were in common with the negative cCRE-gene pairs (p<2.2×10⁻¹⁶, 2,621 observed compared to 185 randomly expected). The cCREs in these shared pairs were preferred to be in the ell-b group, and target genes of whom were enriched for development processes such as gliogenesis and forebrain development. These results are consistent with the recent finding that transition between PRC2-associated silencers and active enhancers occurs during differentiation. Despite the potentially shared fraction, CREs of the repressive pairs are more enriched in intergenic regions as well as are more distal to their targets.

Next, the CREs of different groups were linked with putative target genes based on the predicted pairs. Interestingly, target genes tend to be in the similar group with CREs: for example, target genes of class ell-a and ell-b cCREs were strongly enriched in promoters of class II-a and II-b genes. These genes are enriched in those with functions in development processes. Then, the chromatin state of cCREs were compared with the promoters of the putative target genes: cCREs and promoters from the active pairs displayed higher concordance for their H3K27ac levels, but not for the repressive pairs; on the other hand, higher concordance for H3K27me3 levels was only observed from the repressive pairs. These results support the hypothesis that the distal regulatory elements share similar histone modification states with the promoter regions of their target genes.

Then, the candidate CREs with linked genes were grouped according to their H3K27-methylation and acetylation states. Target genes of neuron-specific cCRE groups are enriched in GO terms including modulation of synaptic transmission, genes linked to cCRE groups of glial cells are enriched for terms including gliogenesis, morphogenesis of epithelium and neuron projection morphogenesis and so on. For the repressive pairs, only a small fraction showed strong cluster-specific enrichment of H3K27me3 and the concordant depletion of gene expression (M12-M14). One of the transcription factors, Sox//, is essential for both embryonic and adult neurogenesis, whose motifs showed a strong H3K27me3 signature in endothelial cells (M14). SOX11 is overexpressed in several solid tumors and is shown to promote endothelial cell proliferation and angiogenesis in aggressive mantle cell lymphomas-derived cell lines. The repressive function of H3K27me3-marked CREs here may restrict the expression levels of Sox11 targets in endothelial cells to maintain proper cell proliferation.

### Example 2

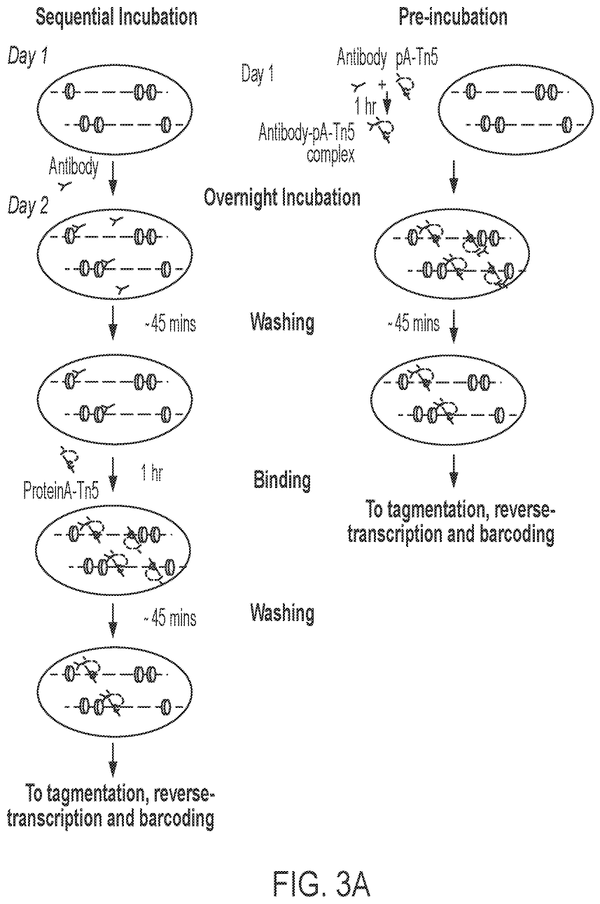

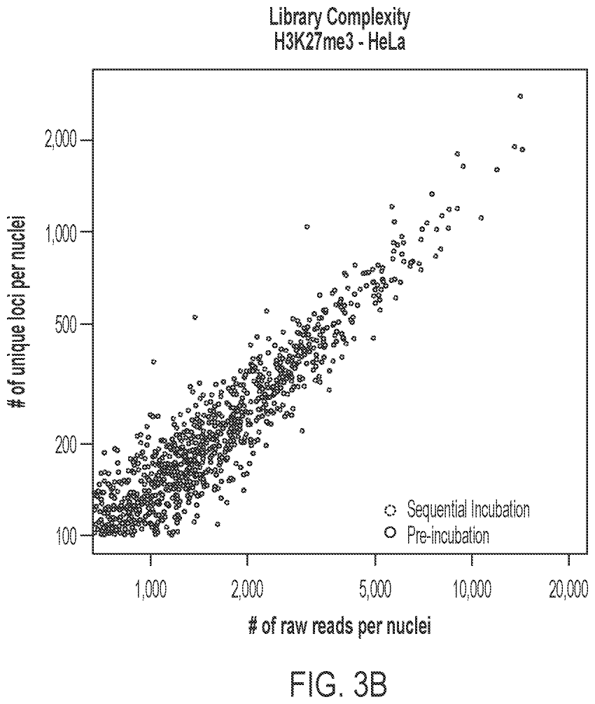

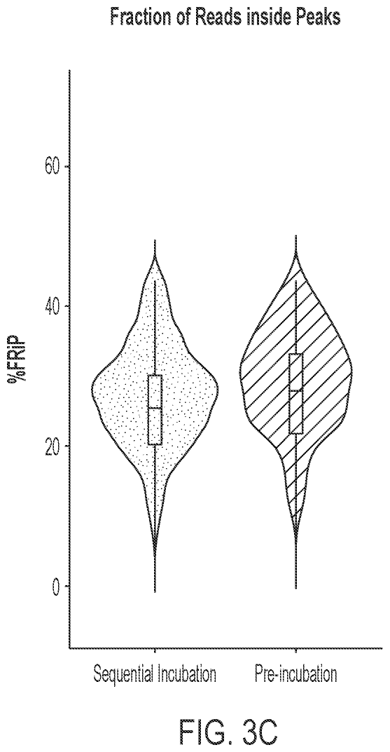

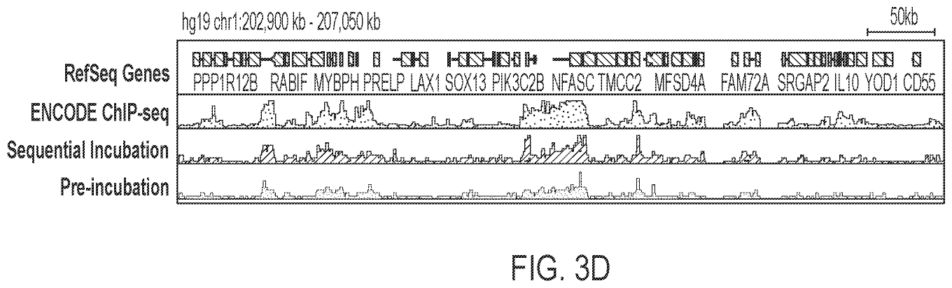

Instead of incubating the nuclei first with the antibody that binds to a chromatin-associated protein or chromatin modification and then incubating the nuclei with pA-Tn5 (FIG. 3A, sequential incubation protocol), pA-Tn5 and antibodies were pre-incubated and the nuclei were subsequently contacted with the Tn⁵/ₐntibody complex (FIG. 3A, pre-incubation protocol). No loss in the quality of the data obtained using the pre-incubation technique as compared with the sequential technique was observed (FIGS. 3B-D).

We claim:

1. A method for obtaining gene expression information for a single nucleus, the method comprising:
a. permeabilizing one or more nuclei;
b. contacting the one or more nuclei with (i) an antibody that binds to a chromatin-associated protein or chromatin modification and (ii) a first transposase;
wherein the first transposase is loaded with a nucleic acid comprising a first tag, wherein the first tag comprises a first restriction site and a barcode selected from a first set of barcodes;
c. initiating a tagmentation reaction, resulting in the generation of genomic DNA fragments comprising the first tag;
d. reverse transcribing the RNA in the one or more nuclei using primers comprising a second tag, wherein the second tag comprising a second restriction site and the barcode of the first tag, resulting in the generation of cDNA comprising the second tag;
e. contacting the one or more nuclei with a ligase and a third tag comprising a second barcode selected from a second set of barcodes, resulting in the generation of genomic DNA fragments comprising a first tag and a third tag and cDNA comprising a second tag and a third tag;
f. lysing the one or more nuclei;
g. fusing a polynucleotide tail to the DNA and cDNA, generating polynucleotide tailed DNA and cDNA;
h. amplifying the polynucleotide tailed DNA and cDNA, wherein one of the primers used for the amplification of the DNA comprises a third restriction site and wherein the third restriction site is recognized by an endonuclease;
i. dividing the amplified polynucleotide tailed DNA and cDNA into a DNA library and an RNA library;
j. for the DNA library:
i. cleaving the amplified polynucleotide tailed DNA with a restriction an endonuclease recognizing the third restriction site;
ii. contacting the DNA end with a sequencing adaptor and a ligase, resulting in the generation of amplified polynucleotide tailed DNA comprising the sequencing adaptor;
iii. cleaving the amplified polynucleotide tailed cDNA with an enzyme recognizing the second restriction site;
k. for the RNA library:
i. cleaving the amplified polynucleotide tailed DNA with a restriction enzyme recognizing the first restriction site;
ii. contacting the amplified polynucleotide tailed cDNA with a second transposase loaded with a nucleic acid comprising a sequencing adaptor and initiating a tagmentation reaction, resulting in the generation of amplified polynucleotide tailed cDNA comprising the sequencing adaptor;
l. sequencing the molecules in the RNA library and the DNA library;
m. correlating the RNA library and the DNA library for each of the one or more nuclei.

2. A method for obtaining gene expression information for a single nucleus, the method comprising:
a. permeabilizing one or more nuclei;
b. contacting the one or more nuclei with (i) an antibody that binds to a chromatin-associated protein or chromatin modification and (ii) a first transposase;
wherein the first transposase is loaded with a nucleic acid comprising a first tag, wherein the first tag comprises a first restriction site and a barcode selected from a first set of barcodes;
c. initiating a tagmentation reaction, resulting in the generation of genomic DNA fragments comprising the first tag;
d. reverse transcribing the RNA in the one or more nuclei using primers comprising a second tag, wherein the second tag comprising a second restriction site and the barcode of the first tag, resulting in the generation of cDNA comprising the second tag;
e. contacting the one or more nuclei with a ligase and a third tag comprising a second barcode selected from a second set of barcodes, resulting in the generation of genomic DNA fragments comprising a first tag and a third tag and cDNA comprising a second tag and a third tag;
f. lysing the one or more nuclei;
g. fusing a polynucleotide tail to the DNA and cDNA, generating polynucleotide tailed DNA and cDNA;
h. amplifying the polynucleotide tailed DNA and cDNA, wherein one of the primers used for the amplification of the cDNA comprises a third restriction site and wherein the third restriction site is recognized by an endonuclease;
i. dividing the amplified polynucleotide tailed DNA and cDNA into a DNA library and an RNA library;
j. for the RNA library:
i. cleaving the amplified polynucleotide tailed cDNA with a restriction an endonuclease recognizing the third restriction site;
ii. contacting the cDNA end with a sequencing adaptor and a ligase, resulting in the generation of amplified polynucleotide tailed cDNA comprising the sequencing adaptor;
iii. cleaving the amplified polynucleotide tailed DNA with an enzyme recognizing the first restriction site;
k. for the DNA library:
i. cleaving the amplified polynucleotide tailed cDNA with a restriction enzyme recognizing the second restriction site;
ii. contacting the amplified polynucleotide tailed DNA with a second transposase loaded with a nucleic acid comprising a sequencing adaptor and initiating a tagmentation reaction, resulting in the generation of amplified polynucleotide tailed DNA comprising the sequencing adaptor;
l. sequencing the molecules in the RNA library and the DNA library;
m. correlating the RNA library and the DNA library for each of the one or more nuclei.

3. A method for obtaining gene expression information for a single nucleus, the method comprising:
a. permeabilizing one or more nuclei;
b. contacting the one or more nuclei with (ii) an antibody that binds to a chromatin-associated protein or chromatin modification and (ii) a first transposase;
wherein the first transposase is loaded with a nucleic acid comprising a first tag, wherein the first tag comprises a first barcode selected from a first set of barcodes;
c. initiating a tagmentation reaction, resulting in the generation of genomic DNA fragments comprising the first tag;
d. reverse transcribing the RNA in the one or more nuclei using primers comprising a second tag, wherein the second tag comprises the barcode of the first tag, resulting in the generation of cDNA comprising the second tag;
wherein the first tag further comprises (i) a first reactive group suitable to perform click chemistry or (ii) a first affinity tag and/or wherein the second tag further comprises (i) a second reactive group suitable to perform click chemistry or (ii) a second affinity tag;
e. contacting the one or more nuclei with a ligase and a third tag comprising a second barcode selected from a second set of barcodes, resulting in the generation of genomic DNA fragments comprising a first tag and a third tag and cDNA comprising a second tag and a third tag;
f. lysing the one or more nuclei;
g. (I) contacting the genomic DNA fragments with an immobilized agent that
(i) reacts with the first reactive group; or
(ii) binds to the first affinity tag; and
performing a pull-down of the genomic DNA to separate the genomic DNA from the cDNA; and/or
(II) contacting the cDNA with an immobilized agent that
(i) reacts with the second reactive group; or
(ii) binds to the second affinity tag; and
performing a pull-down of the cDNA to separate the genomic cDNA from the DNA;
h. for the DNA library:
i. contacting the genomic DNA with random primers comprising a sequencing adaptor, generating polynucleotide tailed DNA; and
ii. amplifying the polynucleotide tailed DNA;
i. for the RNA library:
i. contacting the cDNA with random primers comprising a sequencing adaptor, generating polynucleotide tailed cDNA; and
ii. amplifying the polynucleotide tailed cDNA;
j. sequencing the molecules in the RNA library and the DNA library;
k. correlating the RNA library and the DNA library for each of the one or more nuclei.

4. The method of any one of the preceding claims, wherein in step (b) of the method:
a. the one or more nuclei are first contacted with the antibody and then contacted the first transposase, wherein the first transposase is linked to a binding moiety that binds to the antibody;
b. the antibody is first incubated with the first transposase linked to a binding moiety that binds to the antibody; and the one or more nuclei are contacted with the antibody bound to the transposase;
c. the one or more nuclei are contacted with an antibody that is covalently linked to the first transposase.

5. The method of any one of the preceding claims, the method further comprising after step (e) a step of contacting the one or more nuclei with a ligase and a fourth tag comprising a third barcode selected from a third set of barcodes, resulting in the generation of genomic DNA fragments comprising a first, a third, and a fourth tag and in the generation of cDNA comprising a second, a third tag, and a fourth tag.

6. The method of claim 5, wherein the step of contacting the one or more nuclei with a ligase and a tag comprising an additional barcode is repeated one or more times.

7. A method for obtaining gene expression information for a single nucleus, the method comprising:
a. providing a sample comprising nuclei;
b. dividing the sample into a first set of sub-samples comprising two or more sub-samples;
c. permeabilizing the nuclei in the two or more sub-samples in the first set of sub-samples;
d. contacting the nuclei in the two or more sub-samples in the first set of sub-samples with (i) an antibody that binds to a chromatin-associated protein or chromatin modification and (ii) a first transposase;
wherein the first transposase is loaded with a nucleic acid comprising a first tag comprising a barcode selected from a first set of barcodes;
e. initiating a tagmentation reaction, resulting in the generation of genomic DNA fragments comprising the first tag;
f. reverse transcribing the RNA in the one or more nuclei in the two or more sub-samples in the first set of sub-samples using primers comprising a second tag, wherein the second tag comprising a second restriction site and the barcode of the first tag, resulting in the generated of cDNA comprising the second tag;
g. pooling the first set of sub-samples to generate a first sub-sample pool;
h. dividing the first sub-sample pool into two or more sub-samples to generate a second set of sub-samples;
i. contacting each of the two or more sub-samples in the second set of sub-samples with a ligase and a third tag comprising a barcode selected from a second set of barcodes, wherein the third tag is ligated to the genomic DNA and the cDNA;
j. pooling the second set of sub-samples to generate a second sub-sample pool;
k. dividing the second sub-sample pool into two or more sub-samples to generate a third set of sub-samples;
l. contacting each of the two or more sub-samples in the third set of sub-samples with a ligase and a fourth tag comprising a barcode selected from a third set of barcodes, wherein the fourth tag is ligated to the genomic DNA and the cDNA;
m. pooling the two or more sub-samples in the third set of sub-samples;
n. lysing the nuclei;
o. fusing a polynucleotide tail to the DNA and cDNA, generating polynucleotide tailed DNA and cDNA;
p. amplifying the polynucleotide tailed DNA and cDNA, wherein one of the primers used for the amplification of the DNA comprises a third restriction site;
q. dividing the amplified polynucleotide tailed DNA and cDNA into a DNA library and a RNA library;
r. for the DNA library:
i. cleaving the amplified polynucleotide tailed DNA with a restriction an endonuclease recognizing the third restriction site;
ii. contacting the DNA end with a sequencing adaptor and a ligase, resulting in the generation of amplified polynucleotide tailed DNA comprising the sequencing adaptor;
iii. cleaving the amplified polynucleotide tailed cDNA with an enzyme recognizing the second restriction site;
s. for the RNA library:
i. cleaving the amplified polynucleotide tailed DNA with a restriction enzyme recognizing the first restriction site;
ii. contacting the amplified polynucleotide tailed cDNA with a second transposase loaded with a nucleic acid comprising a sequencing adaptor and initiating a tagmentation reaction, resulting in the generation of amplified polynucleotide tailed cDNA comprising the sequencing adaptor;
t. sequencing the RNA library and the DNA library;
u. correlating the RNA library and the DNA library for each of the one or more nuclei.

8. A method for obtaining gene expression information for a single nucleus, the method comprising:
a. providing a sample comprising nuclei;
b. dividing the sample into a first set of sub-samples comprising two or more sub-samples;
c. permeabilizing the nuclei in the two or more sub-samples in the first set of sub-samples;
d. contacting the nuclei in the two or more sub-samples in the first set of sub-samples with (i) an antibody that binds to a chromatin-associated protein or chromatin modification and (ii) a first transposase;
wherein the first transposase is loaded with a nucleic acid comprising a first tag comprising a barcode selected from a first set of barcodes;
e. initiating a tagmentation reaction, resulting in the generation of genomic DNA fragments comprising the first tag;
f. reverse transcribing the RNA in the one or more nuclei in the two or more sub-samples in the first set of sub-samples using primers comprising a second tag, wherein the second tag comprising a second restriction site and the barcode of the first tag, resulting in the generated of cDNA comprising the second tag;
g. pooling the first set of sub-samples to generate a first sub-sample pool;
h. dividing the first sub-sample pool into two or more sub-samples to generate a second set of sub-samples;
i. contacting each of the two or more sub-samples in the second set of sub-samples with a ligase and a third tag comprising a barcode selected from a second set of barcodes, wherein the third tag is ligated to the genomic DNA and the cDNA;
j. pooling the second set of sub-samples to generate a second sub-sample pool;
k. dividing the second sub-sample pool into two or more sub-samples to generate a third set of sub-samples;
l. contacting each of the two or more sub-samples in the third set of sub-samples with a ligase and a fourth tag comprising a barcode selected from a third set of barcodes, wherein the fourth tag is ligated to the genomic DNA and the cDNA;
m. pooling the two or more sub-samples in the third set of sub-samples;
n. lysing the nuclei;
o. fusing a polynucleotide tail to the DNA and cDNA, generating polynucleotide tailed DNA and cDNA;
p. amplifying the polynucleotide tailed DNA and cDNA, wherein one of the primers used for the amplification of the cDNA comprises a third restriction site;
q. dividing the amplified polynucleotide tailed DNA and cDNA into a DNA library and an RNA library;
r. for the RNA library:
i. cleaving the amplified polynucleotide tailed cDNA with a restriction an endonuclease recognizing the third restriction site;
ii. contacting the cDNA end with a sequencing adaptor and a ligase, resulting in the generation of amplified polynucleotide tailed cDNA comprising the sequencing adaptor;
iii. cleaving the amplified polynucleotide tailed DNA with an enzyme recognizing the first restriction site;
s. for the DNA library:
i. cleaving the amplified polynucleotide tailed cDNA with a restriction enzyme recognizing the second restriction site;
ii. contacting the amplified polynucleotide tailed DNA with a second transposase loaded with a nucleic acid comprising a sequencing adaptor and initiating a tagmentation reaction, resulting in the generation of amplified polynucleotide tailed DNA comprising the sequencing adaptor;
t. sequencing the RNA library and the DNA library;
u. correlating the RNA library and the DNA library for each of the one or more nuclei.

9. The method of claim 7 or 8, wherein in step (d) of the method:
a. the one or more nuclei in the two or more sub-samples are first contacted with the antibody and then contacted the first transposase, wherein the first transposase is linked to a binding moiety that binds to the antibody;
b. the antibody is first incubated with the first transposase linked to a binding moiety that binds to the antibody; and the one or more nuclei in the two or more sub-samples are contacted with the antibody bound to the transposase;
c. the one or more nuclei in the two or more sub-samples are contacted with an antibody that is covalently linked to the first transposase.

10. The method of any one of claims 7-9, wherein after step (m) the steps of pooling;
dividing; and contacting the sub-samples with a ligase and a tag comprising an additional barcode are repeated one or more times.

11. The method of any one of claims 1-2, 4-10, wherein the third restriction site is recognized by a type IIS endonuclease.

12. The method of claim 11, wherein the type IIS endonuclease is selected from the group consisting of FokI, AcuI, AsuHPI, BbvI, BpmI, BpuEI, BseMII, BseRI, BseXI, BsgI, BslFI, BsmFI, BsPCNI, BstV1I, BtgZI, EciI, Eco57I, FaqI, GsuI, HphI, MmeI, NmeAIII, SchI, TaqII, TspDTI, and TspGWI.

13. The method of claims 1-2, 4-12, wherein the polynucleotide tail is fused to the DNA and cDNA by contacting the DNA and cDNA with
(i) a terminal deoxynucleotidyltransferase (TdT);
(ii) a DNA ligase and DNA or RNA oligonucleotide;
(iii) a DNA polymerase and a random primer; or
(iv) a DNA or RNA oligonucleotide with a reactive chemical group that attaches to the 3′-end of the DNA and cDNA.

14. The method of claim 13(ii), wherein the DNA ligase is a T3, T4 or T7 DNA ligase.

15. The method of claim 13(iv), wherein the reactive chemical group is reactive group suitable to perform click chemistry.

16. The method of claim 13(iv), wherein the a reactive chemical group is an azide group or an alkyne group.

17. The method of any one of claim 4-6, or 9-16, wherein the binding moiety linked to the first transposase is protein A.

18. The method of any one of the preceding claims, wherein the chromatin-associated protein is a transcription factor protein is a histone protein, transcription factor, chromatin remodeling complex, RNA polymerase, DNA polymerase, or an accessory protein.

19. The method of any one of the preceding claims, wherein the chromatin modification is a histone modification , DNA modification, RNA modifications, histone variants, or an R-loop.

20. The method of any one of the preceding claims, wherein the nuclei are obtained from a mammal.


SEQUENCE LISTING (53 sequences)

SEQ ID NO: 1 - protein, 142 aa, Staphylococcus aureus
SLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPK

SEQ ID NO: 2 - DNA, 20 bp, Artificial Sequence
  pMENTs
  misc_feature (1)..(1): 5 Phos
  misc_feature (20)..(20): dideoxycytosine
ctgtctcttatacacatctc

SEQ ID NO: 3 - DNA, 33 bp, Artificial Sequence
  AdaptorA
tcgtcggcagcgtcagatgtgtataagagacag

SEQ ID NO: 4 - DNA, 30 bp, Artificial Sequence
  Linker-R02
cgaatgctctggcctctcaagcacgtggat

SEQ ID NO: 5 - DNA, 30 bp, Artificial Sequence
  Blocker-R02
atccacgtgcttgagaggccagagcattcg

SEQ ID NO: 6 - DNA, 30 bp, Artificial Sequence
  Linker-R03
ggtctgagttcgcaccgaaacatcggccac

SEQ ID NO: 7 - DNA, 30 bp, Artificial Sequence
  Quencher-R03
gtggccgatgtttcggtgcgaactcagacc

SEQ ID NO: 8 - DNA, 42 bp, Artificial Sequence
  Anchor-FokI-GH
  misc_feature (41)..(42): phosphorothioate bond modification
aagcagtggtatcaacgcagagtgaaggatgtgggggggggh

SEQ ID NO: 9 - DNA, 33 bp, Artificial Sequence
  P5-FokI
acactctttccctacacgacgctcttccgatct

SEQ ID NO: 10 - DNA, 36 bp, Artificial Sequence
  P5c-NNDC-FokI
  5Phos (1)..(1)
  misc_feature (1)..(2): n is a, c, g, or t
nndcagatcggaagagcgtcgtgtagggaaagagtg

SEQ ID NO: 11 - DNA, 34 bp, Artificial Sequence
  P5H-FokI
acactctttccctacacgacgctcttccgatcth

SEQ ID NO: 12 - DNA, 37 bp, Artificial Sequence
  P5Hc-NNDC-FokI
  misc_feature (1)..(1): 5Phos
  misc_feature (2)..(2): n is a, c, g, or t
nndcdagatcggaagagcgtcgtgtagggaaagagtg

SEQ ID NO: 13 - DNA, 22 bp, Artificial Sequence
  PA-F
cagacgtgtgctcttccgatct

SEQ ID NO: 14 - DNA, 23 bp, Artificial Sequence
  PA-R
aagcagtggtatcaacgcagagt

SEQ ID NO: 15 - DNA, 51 bp, Artificial Sequence
  N5XX
  misc_feature (30)..(37): n is a, c, g, or t
aatgatacggcgaccaccgagatctacacnnnnnnnntcgtcggcagcgtc

SEQ ID NO: 16 - DNA, 63 bp, Artificial Sequence
  P7XX
  misc_feature (25)..(30): n is a, c, g, or t
caagcagaagacggcatacgagatnnnnnngtgactggagttcagacgtgtgctcttccgatc

SEQ ID NO: 17 - DNA, 58 bp, Artificial Sequence
  P5 Universal
  misc_feature (57)..(58): phosphorothioate bond modification
aatgatacggcgaccaccgagatctacactctttccctacacgacgctcttccgatct

SEQ ID NO: 18 - DNA, 47 bp, Artificial Sequence
  DNA_#01_RE
  misc_feature (1)..(1): 5Phos
aggccagagcattcgacatcgcggccgcagatgtgtataagagacag

SEQ ID NO: 19 - DNA, 47 bp, Artificial Sequence
  DNA_#02_RE
  misc_feature (1)..(1): 5Phos
aggccagagcattcgaatgagcggccgcagatgtgtataagagacag

SEQ ID NO: 20 - DNA, 47 bp, Artificial Sequence
  DNA_#03_RE
  misc_feature (1)..(1): 5Phos
aggccagagcattcgaagctgcggccgcagatgtgtataagagacag

SEQ ID NO: 21 - DNA, 47 bp, Artificial Sequence
  DNA_#04_RE
  misc_feature (1)..(1): 5Phos
aggccagagcattcgaacaggcggccgcagatgtgtataagagacag

SEQ ID NO: 22 - DNA, 47 bp, Artificial Sequence
  DNA_#05_RE
  misc_feature (1)..(1): 5Phos
aggccagagcattcgagaatgcggccgcagatgtgtataagagacag

SEQ ID NO: 23 - DNA, 47 bp, Artificial Sequence
  DNA_#06_RE
  misc_feature (1)..(1): 5Phos
aggccagagcattcgatacggcggccgcagatgtgtataagagacag

SEQ ID NO: 24 - DNA, 47 bp, Artificial Sequence
  DNA_#07_RE
  misc_feature (1)..(1): 5Phos
aggccagagcattcgattacgcggccgcagatgtgtataagagacag

SEQ ID NO: 25 - DNA, 47 bp, Artificial Sequence
  DNA_#08_RE
  misc_feature (1)..(1): 5Phos
aggccagagcattcgagttggcggccgcagatgtgtataagagacag

SEQ ID NO: 26 - DNA, 47 bp, Artificial Sequence
  DNA_#09_RE
  misc_feature (1)..(1): 5Phos
aggccagagcattcgaccgtgcggccgcagatgtgtataagagacag

SEQ ID NO: 27 - DNA, 47 bp, Artificial Sequence
  DNA_#10_RE
  misc_feature (1)..(1): 5Phos
aggccagagcattcgacgaagcggccgcagatgtgtataagagacag

SEQ ID NO: 28 - DNA, 47 bp, Artificial Sequence
  DNA_#11_RE
  misc_feature (1)..(1): 5Phos
aggccagagcattcgatctagcggccgcagatgtgtataagagacag

SEQ ID NO: 29 - DNA, 47 bp, Artificial Sequence
  DNA_#12_RE
  misc_feature (1)..(1): 5Phos
aggccagagcattcgagggcgcggccgcagatgtgtataagagacag

SEQ ID NO: 30 - DNA, 46 bp, Artificial Sequence
  RNA_#01_RE
  misc_feature (1)..(1): 5Phos
  misc_feature (45)..(45): v is a or g, or c
  misc_feature (46)..(46): n is a, c, g, or t
aggccagagcattcgtcatccctgcaggttttttttttttttttvn

SEQ ID NO: 31 - DNA, 46 bp, Artificial Sequence
  RNA_#02_RE
  misc_feature (1)..(1): 5Phos
  misc_feature (45)..(45): v is a or g, or c
  misc_feature (46)..(46): n is a, c, g, or t
aggccagagcattcgtatgacctgcaggttttttttttttttttvn

SEQ ID NO: 32 - DNA, 46 bp, Artificial Sequence
  RNA_#03_RE
  misc_feature (1)..(1): 5Phos
  misc_feature (45)..(45): v is a or g, or c
  misc_feature (46)..(46): n is a, c, g, or t
aggccagagcattcgtagctcctgcaggttttttttttttttttvn

SEQ ID NO: 33 - DNA, 46 bp, Artificial Sequence
  RNA_#04_RE
  misc_feature (1)..(1): 5Phos
  misc_feature (45)..(45): v is a or g, or c
  misc_feature (46)..(46): n is a, c, g, or t
aggccagagcattcgtacagcctgcaggttttttttttttttttvn

SEQ ID NO: 34 - DNA, 46 bp, Artificial Sequence
  RNA_#05_RE
  misc_feature (1)..(1): 5Phos
  misc_feature (45)..(45): v is a or g, or c
  misc_feature (46)..(46): n is a, c, g, or t
aggccagagcattcgtgaatcctgcaggttttttttttttttttvn

SEQ ID NO: 35 - DNA, 46 bp, Artificial Sequence
  RNA_#06_RE
  misc_feature (1)..(1): 5Phos
  misc_feature (45)..(45): v is a or g, or c
  misc_feature (46)..(46): n is a, c, g, or t
aggccagagcattcgttacgcctgcaggttttttttttttttttvn

SEQ ID NO: 36 - DNA, 46 bp, Artificial Sequence
  RNA_#07_RE
  misc_feature (1)..(1): 5Phos
  misc_feature (45)..(45): v is a or g, or c
  misc_feature (46)..(46): n is a, c, g, or t
aggccagagcattcgtttaccctgcaggttttttttttttttttvn

SEQ ID NO: 37 - DNA, 46 bp, Artificial Sequence
  RNA_#08_RE
  misc_feature (1)..(1): 5Phos
  misc_feature (45)..(45): v is a or g, or c
  misc_feature (46)..(46): n is a, c, g, or t
aggccagagcattcgtgttgcctgcaggttttttttttttttttvn

SEQ ID NO: 38 - DNA, 46 bp, Artificial Sequence
  RNA_#09_RE
  misc_feature (1)..(1): 5Phos
  misc_feature (45)..(45): v is a or g, or c
  misc_feature (46)..(46): n is a, c, g, or t
aggccagagcattcgtccgtcctgcaggttttttttttttttttvn

SEQ ID NO: 39 - DNA, 46 bp, Artificial Sequence
  RNA_#10_RE
  misc_feature (1)..(1): 5Phos
  misc_feature (45)..(45): v is a or g, or c
  misc_feature (46)..(46): n is a, c, g, or t
aggccagagcattcgtcgaacctgcaggttttttttttttttttvn

SEQ ID NO: 40 - DNA, 46 bp, Artificial Sequence
  RNA_#11_RE
  misc_feature (1)..(1): 5Phos
  misc_feature (45)..(45): v is a or g, or c
  misc_feature (46)..(46): n is a, c, g, or t
aggccagagcattcgttctacctgcaggttttttttttttttttvn

SEQ ID NO: 41 - DNA, 46 bp, Artificial Sequence
  RNA_#12_RE
  misc_feature (1)..(1): 5Phos
  misc_feature (45)..(45): v is a or g, or c
  misc_feature (46)..(46): n is a, c, g, or t
aggccagagcattcgtgggccctgcaggttttttttttttttttvn

SEQ ID NO: 42 - DNA, 34 bp, Artificial Sequence
  RNA_#01_NRE
  misc_feature (1)..(1): 5Phos
  misc_feature (29)..(34): n is a, c, g, or t
aggccagagcattcgtcatccctgcaggnnnnnn

SEQ ID NO: 43 - DNA, 34 bp, Artificial Sequence
  RNA_#02_NRE
  misc_feature (1)..(1): 5Phos
  misc_feature (29)..(34): n is a, c, g, or t
aggccagagcattcgtatgacctgcaggnnnnnn

SEQ ID NO: 44 - DNA, 34 bp, Artificial Sequence
  RNA_#03_NRE
  misc_feature (1)..(1): 5Phos
  misc_feature (29)..(34): n is a, c, g, or t
aggccagagcattcgtagctcctgcaggnnnnnn

SEQ ID NO: 45 - DNA, 34 bp, Artificial Sequence
  RNA_#04_NRE
  misc_feature (1)..(1): 5Phos
  misc_feature (29)..(34): n is a, c, g, or t
aggccagagcattcgtacagcctgcaggnnnnnn

SEQ ID NO: 46 - DNA, 34 bp, Artificial Sequence
  RNA_#05_NRE
  misc_feature (1)..(1): 5Phos
  misc_feature (29)..(34): n is a, c, g, or t
aggccagagcattcgtgaatcctgcaggnnnnnn

SEQ ID NO: 47 - DNA, 34 bp, Artificial Sequence
  RNA_#06_NRE
  misc_feature (1)..(1): 5Phos
  misc_feature (29)..(34): n is a, c, g, or t
aggccagagcattcgttacgcctgcaggnnnnnn

SEQ ID NO: 48 - DNA, 34 bp, Artificial Sequence
  RNA_#07_NRE
  misc_feature (1)..(1): 5Phos
  misc_feature (29)..(34): n is a, c, g, or t
aggccagagcattcgtttaccctgcaggnnnnnn

SEQ ID NO: 49 - DNA, 34 bp, Artificial Sequence
  RNA_#08_NRE
  misc_feature (1)..(1): 5Phos
  misc_feature (29)..(34): n is a, c, g, or t
aggccagagcattcgtgttgcctgcaggnnnnnn

SEQ ID NO: 50 - DNA, 34 bp, Artificial Sequence
  RNA_#09_NRE
  misc_feature (1)..(1): 5Phos
  misc_feature (29)..(34): n is a, c, g, or t
aggccagagcattcgtccgtcctgcaggnnnnnn

SEQ ID NO: 51 - DNA, 34 bp, Artificial Sequence
  RNA_#10_NRE
  misc_feature (1)..(1): 5Phos
  misc_feature (29)..(34): n is a, c, g, or t
aggccagagcattcgtcgaacctgcaggnnnnnn

SEQ ID NO: 52 - DNA, 34 bp, Artificial Sequence
  RNA_#11_NRE
  misc_feature (1)..(1): 5Phos
  misc_feature (29)..(34): n is a, c, g, or t
aggccagagcattcgttctacctgcaggnnnnnn

SEQ ID NO: 53 - DNA, 34 bp, Artificial Sequence
  RNA_#12_NRE
  misc_feature (1)..(1): 5Phos
  misc_feature (29)..(34): n is a, c, g, or t
aggccagagcattcgtgggccctgcaggnnnnnn